(19) United States
(12) Patent Application Publication
(10) Pub. No.: US 2022/0370559 A1
(43) Pub. Date: Nov. 24, 2022

(54) METHOD OF TREATMENT OF CANCER OR TUMOUR

(71) Applicant: Adaptimmune Limited, Abingdon Oxfordshire (GB)

(72) Inventors: Jonathan David Silk, Abingdon Oxfordshire (GB); Emily Puo San Lam, Abingdon Oxfordshire (GB); Alexandra Sevko, Abingdon Oxfordshire (GB); Dennis Robert Williams, Abingdon Oxfordshire (GB); Samik Basu, Abingdon Oxfordshire (GB)

(21) Appl. No.: 17/812,304

(22) Filed: Jul. 13, 2022

Related U.S. Application Data
(63) Continuation of application No. PCT/EP2021/050641, filed on Jan. 14, 2021.

(30) Foreign Application Priority Data
Jan. 14, 2020 (GB) 2000546.8
May 1, 2020 (GB) 2006474.7

(51) Int. Cl. A61K 38/17; A61K 35/12; A61K 9/00; A61P 35/00

(52) U.S. Cl. CPC A61K 38/1774; A61K 35/12; A61K 9/0019; A61P 35/00

(57) ABSTRACT

The present invention provides methods of treating, preventing or delaying the progress of cancer and/or tumour in a subject comprising administering to the subject a treatment regimen comprising an effective amount of a PD-1 axis binding antagonist and a population of modified immunoresponsive cells expressing or presenting a heterologous TCR. The invention also provides methods of enhancing immune function in a subject having cancer and/or tumour comprising administering to the subject a treatment regimen comprising an effective amount of a PD-1 axis binding antagonist and a population of modified immunoresponsive cells expressing or presenting a heterologous TCR.

### CROSS-REFERENCE TO RELATED PATENT APPLICATIONS

This application is a continuation application of International Application No. PCT/EP2021/050641, filed Jan. 14, 2021, which claims the priority to Great Britain Patent Application Nos. 2000546.8, filed Jan. 14, 2020, and 2006474.7, filed May 1, 2020, the contents of each of which are hereby incorporated by reference in their entirety into the present disclosure.

### REFERENCE TO AN ELECTRONIC SEQUENCE LISTING

The contents of the electronic sequence listing (PC10112A.xml; Size: 57,344 bytes; and Date of Creation: Jul. 12, 2022) is herein incorporated by reference in its entirety.

### FIELD OF INVENTION

The present invention relates to the treatment of cancer or tumour by the administration of a combination of a PD-1 axis binding antagonist, such as an anti-PD1 or anti-PD-L1 antibody and an immunoresponsive cell such as a T cell presenting a heterologous T cell receptor (TCR) or Chimeric Antigen Receptor (CAR).

### BACKGROUND

Immune tolerance, T cell exhaustion and functional suppression in the tumour micro-environment is a challenge to cancer immunotherapy, one objective for the successful treatment of cancer is to seek therapies aimed at breaking tolerance within the tumor microenvironment and enhancing and maintaining T cell activation.

The antitumor immune response starts with tumor antigen release and uptake by dendritic cells (DC) which process the antigen and present it via MHC to T cells presenting antigen-specific T cell receptor, this stimulation of the T cells results in their activation and proliferation. An effective antitumor immune response requires the maintenance of the activated T cell response in order to effectively eliminate the tumour. The problem persists that the tumor microenvironment is rendered immunosuppressive by the tumor cells themselves and suppressive immune populations of myeloid-derived suppressor cells (MDSCs) and Regulatory T cells (Tregs); additionally immune tolerance can result from suppression at any of the key stages of the antitumor immune response, i.e. the dendritic cell; antigen presentation, the major histocompatibility complex; priming, signaling, activation or trafficking of T cell or T cell penetration into the tumour, the T cell receptor; MDSC, Tregs, and cancer-associated fibroblasts, that promote higher levels of inhibitory ligands and cytokines. This problem may lead to lack of patient response to cancer therapy by adoptive transfer of immune effectors such as antitumor monoclonal antibodies, Chimeric Antigen Receptor (CAR) T cells, TCR engineered T cells or patient relapse following treatment despite having measurable antibody or transduced T cells in circulation. Some cancers such as esophageal cancer, stomach cancer, liver cancer, bile duct cancer, pancreatic cancer, head and neck cancer, lung cancer, ovarian cancer, breast cancer appear to pose particular problems in this regard.

Primary and acquired resistance is additionally an increasing challenge to effective cancer and tumour therapy with checkpoint inhibitors, for example PD-1 axis binding antagonist therapy with anti-PD1 or anti-PD-L1 antibody. A variety of mechanisms are thought to underlie such resistance, tumour micro-environment suppressors, low tumour immunogenicity, PD1 resistance of tumour, T-cell infiltration or exclusion mechanisms, tumour resistance to interferons and upregulation of additional checkpoint inhibitor receptors by the T-cell. Some cancers appear highly resistant checkpoint inhibitor treatment, partial patient response, non-response and patients developing acquired resistance to check point inhibitor treatment is a significant problem in cancer therapy.

There is therefore a need to develop therapies that address the primary and acquired resistance to checkpoint inhibitor therapy of cancer and tumour and that improve T cell function, enhance T cell survival persistence and efficacy, activate the immune system and/or overcome the local immunosuppressive tumour microenvironment.

The present invention relates to the combination treatment of cancer and/or tumour comprising a PD-1 axis binding antagonist and a population of modified immunoresponsive cells expressing or presenting a heterologous TCR or CAR, the combination treatment results in enhancement of immune response to cancer or tumour.

### SUMMARY OF THE INVENTION

The present invention relates to a combination treatment of cancer and/or tumour comprising a PD-1 axis binding antagonist and a population of modified immunoresponsive cells expressing or presenting a heterologous TCR or CAR. Preferably the combination treatment results in enhancement of immune response to cancer or tumour.

The present invention provides a method of treating, preventing or delaying the progression of cancer and/or tumour in a subject comprising administering to the subject an effective amount of a PD-1 axis binding antagonist and a population of modified immunoresponsive cells expressing or presenting a heterologous TCR or CAR. Also provided is a method of enhancement of immune response to cancer and/or tumour in a subject comprising administering to the subject an effective amount of a PD-1 axis binding antagonist and a population of modified immunoresponsive cells expressing or presenting a heterologous TCR or CAR.

The present invention also provides a PD-1 axis binding antagonist in combination with a population of modified immunoresponsive cells expressing or presenting a heterologous TCR or CAR, for use in treating, preventing or delaying the progression of cancer and/or tumour in a subject or for use in the enhancement of immune response to cancer and/or tumour.

The present invention further provides the use of a PD-1 axis binding antagonist in combination with a population of modified immunoresponsive cells expressing or presenting a heterologous TCR or CAR, in the manufacture of a medicament for treating, preventing or delaying the progression of cancer and/or tumour in a subject or for the enhancement of immune response to cancer and/or tumour.

Also provided by the invention herein is a manufacturing process of a medicament or of medicaments for treating, preventing or delaying the progression of cancer and/or tumour in a subject or for the enhancement of immune response to cancer or tumour, comprising the use of a PD-1 axis binding antagonist and a population of modified immunoresponsive cells expressing or presenting a heterologous TCR or CAR.

Also provided is a method of treating, preventing or delaying the progression of cancer and/or tumour in a subject comprising administering to the subject a treatment regimen comprising an effective amount of a PD-1 axis binding antagonist and a population of modified immunoresponsive cells expressing or presenting a heterologous TCR or CAR.

The present invention also provides a PD-1 axis binding antagonist for use in a method of treating, preventing or delaying the progression of cancer and/or tumour in a subject the method comprising administering to the subject a treatment regimen comprising an effective amount of a PD-1 axis binding antagonist and a population of modified immunoresponsive cells expressing or presenting a heterologous TCR or CAR.

Also provided is a method for the enhancement of immune response to cancer and/or tumour in a subject comprising administering to the subject a treatment regimen comprising an effective amount of a PD-1 axis binding antagonist and a population of modified immunoresponsive cells expressing or presenting a heterologous TCR or CAR.

The present invention also provides a PD-1 axis binding antagonist for use in a method for the enhancement of immune response to cancer and/or tumour in a subject the method comprising administering to the subject a treatment regimen comprising an effective amount of a PD-1 axis binding antagonist and a population of modified immunoresponsive cells expressing or presenting a heterologous TCR or CAR.

According to the invention the treatment results in a complete response, a partial response, or stable disease in the subject; and/or a sustained response in the subject either during treatment or after cessation of the treatment; optionally improved relative to prior to administration of the treatment or relative to treatment with the PD-1 axis binding antagonist alone or modified immunoresponsive cells alone.

The PD-1 Axis Binding Antagonist

According to the invention the PD-1 axis binding antagonist is a molecule that inhibits the interaction of a PD-1 axis binding partner with its cognate binding partner or decreases, prevents or inhibits the signal transduction resulting from the interaction of PD-1 with one or more of its binding partners, such as for example PD-L1, PD-L2. The PD-1 axis binding antagonist can diminish, inhibit or prevent the PD1 axis mediated inhibitory effects on T-cells or overcome T-cell dysfunction or exhaustion, for example with a result being to restore or enhance T-cell function, for example as indicated by T-cell proliferation, cytokine production, target cell killing, activation, CD28 signalling, ability to infiltrate tumour, to recognise and bind to dendritic cell presented antigen and/or to produce interferon.

According to the invention the PD-1 axis binding antagonist can be an antagonist in the sense that it antagonizes, blocks, inhibits or reduces the biological activity of the antigen, for example PD-1 related target, it binds. Hence, the PD-1 axis binding antagonist may antagonise, block, inhibit or reduce the signalling through PD-1 so as to restore a functional response by T-cells, such as for example T-cell proliferation, cytokine production, target cell killing, and thereby rescuing the T-cell from a dysfunctional state to antigen stimulation, and enhancing T-cell function for example by overcoming T-cell exhaustion, anergy, dysfunction, tumour immunity, enhancing tumour immunogenicity or improved sustained response.

According to the present invention the PD-1 axis binding antagonist can be (a) a PD-1 antagonist and/or binding antagonist, (b) a PD-L1 antagonist and/or binding antagonist, (c) a PD-L2 antagonist and/or binding antagonist. The PD-1 axis binding antagonist can bind PD-1 and/or bind to SEQ ID NO: 1 or (b) bind PD-L1 and/or bind to SEQ ID NO: 2.

According to the present invention the PD-1 axis binding antagonist can (a) inhibit the binding of PD-1 to its ligand binding partners, (b) inhibit the binding of PD-L1 to its ligand binding partners.

Accordingly, where the PD-1 axis binding antagonist is a PD-1 binding antagonist it can inhibit the binding of PD-1 to one or more of; (a) PD-L1, (b) PD-L2, or (c) both PD-L1 and PD-L2. The PD-1 binding antagonist may include anti-PD-1 antibodies, antigen binding fragments thereof, immunoadhesins, fusion proteins and oligopeptides that inhibit, decrease, prevent, or interfere with signal transduction due to the interaction of PD-1 with PD-L1 and/or PD-L2. The PD-1 binding antagonist may reduce the negative co-stimulatory signal mediated by or through cell surface proteins expressed on T lymphocytes and/or mediated signalling through PD-1 so as render a dysfunctional T-cell less dysfunctional and thereby enhance T-cell effector function in response to antigen and/or enhancing T-cell function for example by overcoming T-cell exhaustion, anergy, dysfunction, tumour immunity, enhancing tumour immunogenicity and/or improved sustained response.

Accordingly, where the PD-1 axis binding antagonist is a PD-L1 binding antagonist it may inhibit the binding of PD-L1 to one or more of its binding partners, such as (a) PD1, (b) CD80, (c) B7-1 and/or decrease, inhibit, prevent or interfere with signal transduction resulting from the interaction of PD-L1 with either one or more of its binding partners. The PD-L1 binding antagonist may include anti-PD-L1 antibodies, antigen binding fragments thereof, immunoadhesins, fusion proteins, or oligopeptides and/or may reduce the negative co-stimulatory signal mediated by or through cell surface proteins expressed on T lymphocytes and/or mediated signalling through PD-L1 so as to render a dysfunctional T-cell less dysfunctional and thereby enhance T-cell effector response to antigen and/or enhancing T-cell function for example by overcoming T-cell exhaustion, anergy, dysfunction, tumour immunity, enhancing tumour immunogenicity or improved sustained response.

Accordingly, where the PD-1 axis binding antagonist can be a PD-L2 binding antagonist it can bind to PD-L2 to and/or bind to SEQ ID NO: 3 and/or it can inhibit the binding of PD-L2 to one or more of its binding partners, such as PD-1 and/or decrease, inhibit, prevent or interfere with signal transduction resulting from the interaction of PD-L2 with either one or more of its binding partners. The PD-L2 binding antagonist may include anti-PD-L2 antibodies, antigen binding fragments thereof, immunoadhesins, fusion proteins, or oligopeptides and/or may reduce the negative co-stimulatory signal mediated by or through cell surface proteins expressed on T lymphocytes mediated signalling through PD-L2 so as to render a dysfunctional T-cell less dysfunctional and thereby enhance T-cell effector response to antigen.

According to the present invention the PD-1 axis binding antagonist, the PD-1 binding antagonist or PD-L1 binding antagonist can be an antibody; optionally wherein

(a) the anti-PD-L1 antibody inhibits binding between PD-L1 and PD-1 and/or between PD-L1 and B7-1,

(b) the anti-PD-L1 antibody inhibits PD-L1 on the cancer cell surface from transducing a signal to the intracellular pathway,

(c) the anti-PD-1 antibody inhibits binding between PD-L1 and PD-1 and/or between PD-L2 and PD-1,

(d) the anti-PD-1 antibody inhibits PD-1 on the T cell surface from transducing a signal to the intracellular pathway. In the foregoing the intracellular pathway may be the T-cell intracellular pathway, stimulation of which may render the T-cell dysfunctional and thereby diminishing T-cell effector function in response to antigen.

According to the present invention the PD-L1 binding antagonist may be selected from; (a) Durvalumab, Imfinzi or MEDI4736, (b) Atezolizumab, Tecentriq or MPDL3280A, (c) Avelumab, Bavencio or MSB0010718C, (d) MDX-1105, BMS-936559.

Accordingly, the PD-L1 binding antagonist may be an antibody comprising:

(a) a heavy chain comprising SEQ ID NO:4, and a light chain comprising SEQ ID NO:5, or variable regions thereof or CDRs thereof,

(b) a heavy chain comprising SEQ ID NO:6, and a light chain comprising SEQ ID NO:7, or variable regions thereof or CDRs thereof,

(c) a heavy chain comprising SEQ ID NO:8, and a light chain comprising SEQ ID NO:9, or variable regions thereof or CDRs thereof, or

(d) a heavy chain comprising SEQ ID NO:10, and a light chain comprising SEQ ID NO:11, or variable regions thereof or CDRs thereof; or

(e) an antigen binding region of any of (a) to (d).

According to the present invention the PD-1 binding antagonist may be selected from; (a) Pembrolizumab, Keytruda, Lambrolizumab or MK-3475, (b) Cemiplimab, Libtayo, or REGN-2810, (c) BMS/ONO, Nivolumab, Opdivo, ONO-4538, BMS-936558 or MDX1106.

Accordingly, the PD-1 binding antagonist may be an antibody comprising:

(a) a heavy chain comprising SEQ ID NO:12, and a light chain comprising SEQ ID NO:13, or variable regions thereof or CDRs thereof,

(b) a heavy chain comprising SEQ ID NO:14, and a light chain comprising SEQ ID NO:15, or variable regions thereof or CDRs thereof, or

(c) a heavy chain comprising SEQ ID NO:16, and a light chain comprising SEQ ID NO: 17, or variable regions thereof or CDRs thereof; or

(d) an antigen binding region of any of (a) to (c).

According to the present invention the PD-1 axis binding antagonist, the PD-1 or PD-L1 or PD-L2 binding antagonist may be an antibody; for example the antibody may be monocloncal, human or humanised, a full length or antigen-binding fragment thereof, for example Fv, Fab, Fab′, Fab′-SH, F(ab′)2; diabody; linear antibody; single-chain antibody molecule, or scFv. The antibody isotype may be selected from any of the five classes of immunoglobulins: IgA, IgD, IgE, IgG and IgM, having heavy chains designated alpha, delta, epsilon, gamma and mu (M), respectively. The gamma and alpha class antibodies may be of any of subclasses: IgG1, IgG2A, IgG2B, IgG3, IgG4, IgA1 and IgA2.

According to the invention the PD-1 axis binding antagonist, which may be a PD-1 binding, PD-L1 binding antagonist, or P2-L2 binding antagonist, can be an immunoadhesin. The immunoadhesin may comprise an adhesin domain comprising binding specificity, for example the binding site of a receptor or a ligand, with an immunoglobulin constant domain, for example from any isotype such as IgG-1, IgG-2, IgG2A, IgG2B, IgG-3, or IgG-4 subtypes, IgA, IgA-1, IgA-2, IgE, IgD or IgM and/or may comprise the hinge, CH2 and CH3, or the hinge, CH 1, CH2 and CH3 regions of an immunoglobulin molecule. Accordingly the immunoadhesin may be polypeptides that comprise the extracellular or PD-1 binding portions of PD-L1 or PD-L2 or the extracellular or PD-L1 or PD-L2 binding portions of PD-1, fused to a constant domain of an immunoglobulin sequence, such as a PD-L1 ECD-Fc, a PD-L2 ECD-Fc, and a PD-1 ECD-Fc.

Immunoresponsive Cells

According to the present invention the modified immunoresponsive cells can be cells of the lymphoid lineage, comprising B, T or natural killer (NK) cells. The modified immunoresponsive cells may be cells of the lymphoid lineage including T cells, Natural Killer T (NKT) cells, and precursors thereof including embryonic stem cells, and pluripotent stem cells (e.g, those from which lymphoid cells may be differentiated). T cells can be lymphocytes that mature in the thymus and are chiefly responsible for cell-mediated immunity and also involved in the adaptive immune system. According to the present invention the T cells can include, but are not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and gamma-delta T cells. Cytotoxic T cells (CTL or killer T cells) are a subset of T-lymphocytes capable of inducing the death of infected somatic or tumour cells. A subject's own T cells may be genetically modified to target specific antigens through the introduction of a TCR. Preferably, the modified immunoresponsive cell is a T cell optionally a CD4⁺T cell or a CD8⁺T cell. Accordingly the modified immunoresponsive cells may be T-cells, optionally CD4+ T cells or CD8+ T cells, or the modified immunoresponsive cells may be a population of modified T-cells, optionally CD4+ T cells; or CD8+ T cells, or a mixed population of CD4+ T cells and CD8+ T cells.

Heterologous TCR/CAR

According to the present invention the modified immunoresponsive cells can express a heterologous T cell receptor (TCR) or heterologous chimeric antigen receptor (CAR). Upon binding to the antigen, the modified immunoresponsive cells can exhibit T cell effector functions and/or cytolytic effects towards cells bearing the antigen and/or undergo proliferation and/or cell division. In certain embodiments, the modified immunoresponsive cells comprising the TCR exhibits comparable or better therapeutic potency compared to cells comprising a chimeric antigen receptor (CAR) targeting the same cancer and/or tumour antigen and/or peptide (antigenic peptide). Activated modified immunoresponsive cells comprising the TCR or CAR can secrete anti-tumor cytokines which can include, but are not limited to, TNFalpha, IFNy and IL2.

According to the invention the modified immunoresponsive cells may comprise a nucleic acid, construct or vector, or heterologous nucleic acid, construct or vector, encoding a heterologous T cell receptor (TCR) or heterologous chimeric antigen receptor (CAR). Optionally the TCR may be an affinity enhanced TCR, for example a specific peptide enhanced affinity receptor (SPEAR) TCR.

The term “heterologous” or “exogenous” refers to a polypeptide or nucleic acid that is foreign to a particular biological system, such as a cell or host cell, and is not naturally present in that system and which may be introduced to the system by artificial or recombinant means. Accordingly, the expression of a TCR or CAR which is heterologous, may thereby alter the immunogenic specificity of the T cells so that they recognise or display improved recognition for one or more tumour or cancer antigens and/or peptides that are present on the surface of the cancer cells of an individual with cancer. The modification of T cells and their subsequent expansion may be performed in vitro and/or ex vivo.

Cancer Antigen

According to the present invention the cancer and/or tumour antigen or peptide antigen thereof may be a cancer-testis antigen, NY-ESO-1, MART-1 (melanoma antigen recognized by T cells), WT1 (Wilms tumor 1), gp100 (glycoprotein 100), tyrosinase, PRAME (preferentially expressed antigen in melanoma), p53, HPV-E6/HPV-E7 (human papillomavirus), HBV, TRAIL, DR4, Thyroglobin, TGFBII frameshift antigen, LAGE-1A, KRAS, CMV (cytomegalovirus), CEA (carcinoembryonic antigen), AFP (α-fetoprotein), MAGE-AL MAGE-A2, MAGE-A3, MAGE-A4, MAGE-A6, MAGE-A8, and MAGE-A9, MAGE-A10, or MAGE-A12, or peptide antigen thereof. According to the present invention preferably the tumour antigen is MAGE-A4, for example SEQ ID NO: 36 or peptide antigen thereof. Preferably the cancer and/or tumour antigen peptide has the amino acid sequence GVYDGREHTV, SEQ ID NO: 18.

Co-Stimulatory Ligand

According to the present invention, the modified immunoresponsive cells, may further comprise an exogenous or a recombinant (e.g., the cell is transduced with) at least one co-stimulatory ligand, optionally one, two, three or four. The modified immunoresponsive cells, may co-express the TCR or CAR and the at least one exogenous co-stimulatory ligand. The interaction between the TCR or CAR and at the least one exogenous co-stimulatory ligand may provide a non-antigen-specific signal and activation of the cell. Co-stimulatory ligands include, but are not limited to, members of the tumour necrosis factor (TNF) superfamily, and immunoglobulin (Ig) superfamily ligands. TNF is a cytokine involved in systemic inflammation and stimulates the acute phase reaction. TNF superfamily members include, but are not limited to, nerve growth factor (NGF), CD40L (CD40L)/CD154, CD137L/4-1BBL, TNF-alpha, CD134L/OX40L/CD252, CD27L/CD70, Fas ligand (FasL), CD30L/CD153, tumour necrosis factor beta (TNFP)/lymphotoxin-alpha (LTa), lymphotoxin-beta (TTb), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold) Immunoglobulin superfamily ligands include, but are not limited to, CD80 and CD86, both ligands for CD28. In certain embodiments, the at least one co-stimulatory ligand is selected from the group consisting of 4-1BBL, CD275, CD80, CD86, CD70, OX40L, CD48, TNFRSF14, and combinations thereof. The at least one exogenous or recombinant co-stimulatory ligand can be 4-1BBL or CD80, preferably, the at least one exogenous or recombinant co-stimulatory ligand is 4-1BBL. The modified immunoresponsive cells may comprise two exogenous or recombinant co-stimulatory ligands, preferably the two exogenous or recombinant co-stimulatory ligands are 4-1BBL and CD80.

The modified immunoresponsive cells may comprise an exogenous or a recombinant (e.g., the cell is transduced with) at least one construct which overcomes the immunosuppressive tumour microenvironment. Such constructs can be, but are not limited to, cyclic AMP phosphodiesterases and dominant-negative transforming growth factor beta (TGFbeta) receptor II. The modified immunoresponsive cell, modified T cell or a population of modified T cells may be engineered to release cytokines which have a positive effect on the cytolytic activity of said cells. Such cytokines include, but are not limited to interleukin-7, interleukin-15 and interleukin-21.

Specific Binding TCR/CAR

According to the invention the modified immunoresponsive cells, for example modified T cells, may be modified to express a heterologous TCR or CAR, which binds or specifically binds to tumour cells and/or tissue and/or cancer cells and/or tissue of a subject, patient or cancer patient suffering from a disease condition or cancerous condition. The subject, patient or cancer patient may be subsequently treated with the modified immunoresponsive cells or modified T cells or population thereof according to the invention. Suitable cancer patients for treatment according to the invention with the modified immunoresponsive cells or modified T cells may be identified by a method comprising; obtaining sample of tumour and/or cancer cells from an individual or subject with tumour and/or cancer and; identifying the tumour and/or cancer cells cells as binding to the TCR or CAR expressed by the modified immunoresponsive cells.

According to the invention the heterologous TCR or CAR binds or specifically binds to a cancer and/or tumour antigen or peptide antigen thereof. According to the invention the heterologous TCR or CAR binds or specifically binds to a cancer and/or tumour antigen or peptide antigen thereof associated with a cancerous condition and/or presented by tumour or cancer cell or tissue.

Specificity describes the strength of binding between the heterologous TCR or CAR and a specific target cancer and/or tumour antigen or peptide antigen thereof and may be described by a dissociation constant, Kd, the ratio between bound and unbound states for the receptor-ligand system. Additionally, the fewer different cancer and/or tumour antigens or peptide antigen thereof the heterologous TCR or CAR can bind, the greater its binding specificity.

According to the invention the heterologous TCR or CAR may bind to less than 10, 9, 8, 7, 6, 5, 4, 3, 2 different cancer and/or tumour antigens or peptide antigen thereof, for example to only one cancer and/or tumour antigens or peptide antigen thereof.

According to the invention the heterologous TCR or CAR may bind with a dissociation constant of between, 0.01 μM and 100 μM, between 0.01 μM and 50 μM, between 0.01 μM and 20 μM, between 0.05 μM and 20 μM or of 0.01, 0.02, 0.03, 0.04, 0.05, 0.06, 0.07, 0.08, 0.09, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, 10.0 μM; or between 10 μM and 1000 μM, between 10 μM and 500 μM, between 50 μM and 500 μM or of 10, 20 30, 40, 50 60, 70, 80, 90, 100 μM, 150 μM, 200 μM, 250 μM, 300 μM, 350 μM, 400 μM, 450 μM, 500 μM; optionally measured with surface plasmon resonance, optionally at 25° C., optionally between a pH of 6.5 and 6.9 or 7.0 and 7.5. The dissociation constant, K_{D} or k_{off}/kₒₙ may be determined by experimentally measuring the dissociation rate constant, k_{off}, and the association rate constant, kₒₙ. A TCR dissociation constant may be measured using a soluble form of the TCR, wherein the TCR comprises a TCR alpha chain variable domain and a TCR beta chain variable domain. Accordingly, a heterologous TCR or CAR for use in accordance with the invention is capable of binding efficiently and/or with high affinity to HLA displaying GVYDGREHTV, optionally in complex with a peptide presenting molecule for example an HLA, for example with HLA-A*02 or HLA-A*0201, alternatively without presentation in complex with a peptide presenting molecule, for example with a dissociation constant of between 0.01 μM and 100 μM such as 50 μM, 100 μM, 200 μM, 500 μM, preferably between 0.05 μM to 20.0 μM.

According to the present invention the modified immune-responsive cells, for example modified T-cells, may comprise a heterologous TCR or CAR which may bind, specifically bind and/or bind with high affinity to a cancer and/or tumour antigen or peptide antigen thereof optionally associated with a cancerous condition and/or presented by tumour of cancer cell or tissue; optionally wherein the cancer and/or tumour antigen or peptide antigen thereof is recognised by the heterologous TCR or CAR, optionally in complex with a peptide presenting molecule for example an HLA, for example with HLA-A*02 or HLA-A*0201, alternatively without presentation in complex with a peptide presenting molecule for example or HLA.

According to the present invention the heterologous T cell receptor (TCR) or CAR, and modified immunoresponsive cells comprising the heterologous T cell receptor (TCR) or CAR may have the property of binding to an endogenously expressed tumour cell surface a cancer and/or tumour antigen or peptide antigen thereof optionally wherein the binding is independent of presentation of the cell surface antigen as a complex with an peptide-presenting or antigen-presenting molecule, for example major histocompatibility complex (MHC) or human leukocyte antigen (HLA) or major histocompatibility complex class related protein (MR)1.

According to the present invention the TCR or CAR binding may be specific for one cancer and/or tumour antigen, for example a MAGE protein such as MAGE A4, or peptide antigen thereof optionally in comparison to a closely related cancer and/or tumour antigen or peptide antigen sequence. The closely related cancer and/or tumour antigen or peptide antigen sequence may be of similar or identical length and/or may have a similar number or identical number of amino acid residues. The closely related peptide antigen sequence may share between 50 or 60 or 70 or 80 to 90% identity, preferably between 80 to 90% identity and/or may differ by 1, 2, 3 or 4 amino acid residues. The closely related peptide sequence may be derived from the polypeptide sequence of sequence GVYDGREHTV, SEQ ID NO: 18.

The binding affinity may be determined by equilibrium methods (e.g. enzyme-linked immunosorbent assay (ELISA) or radioimmunoassay (RIA)), or kinetics (e.g. BIACORE™ analysis). Avidity is the sum total of the strength of binding of two molecules to one another at multiple sites, e.g. taking into account the valency of the interaction. According to the invention the immunoresponsive cells may demonstrate improved affinity and/or avidity to a cancer and/or tumour antigen or peptide antigen thereof, or a cancer and/or tumour antigen or peptide antigen thereof presented by tumour or cancer cell or tissue and recognised by the heterologous TCR or CAR in comparison immunoresponsive cells lacking the heterologous TCR or CAR or having an alternative heterologous TCR or CAR.

Selective Binding TCR/CAR

According to the invention, the heterologous TCR or CAR may selectively bind to a cancer and/or tumour antigen or peptide antigen thereof, optionally associated with a cancerous condition and/or presented by tumour or cancer cell or tissue; optionally wherein the cancer and/or tumour antigen or peptide antigen thereof is recognised by the heterologous TCR or CAR, optionally in complex with a peptide presenting molecule for example an HLA, for example with HLA-A*02 or HLA-A*0201, alternatively without presentation in complex with a peptide presenting molecule or HLA, preferably expressed by a tumour cell or a cancer cell or tissue.

Selective binding denotes that the heterologous TCR or CAR binds with greater affinity to one cancer and/or tumour antigen or peptide antigen thereof in comparison to another. Selective binding is denoted by the equilibrium constant for the displacement by one ligand antigen of another ligand antigen in a complex with the heterologous TCR or CAR.

Specific/Selective Binding TCR/CAR

According to the present invention the heterologous TCR or CAR binding is selective and/or specific for a cancer and/or tumour antigen or peptide antigen thereof which may be a cancer-testis antigen, NY-ESO-1, MART-1 (melanoma antigen recognized by T cells), WT1 (Wilms tumor 1), gp100 (glycoprotein 100), tyrosinase, PRAME (preferentially expressed antigen in melanoma), p53, HPV-E6/HPV-E7 (human papillomavirus), HBV, TRAIL, DR4, Thyroglobin, TGFBII frameshift antigen, LAGE-1A, KRAS, CMV (cytomegalovirus), CEA (carcinoembryonic antigen), AFP (α-fetoprotein), MAGE-AL MAGE-A2, MAGE-A3, MAGE-A4, MAGE-A6, MAGE-A8, and MAGE-A9, MAGE-A10, or MAGE-A12, or peptide antigen thereof. Preferably the tumour antigen is MAGE-A4 or peptide antigen thereof.

According to the present invention the heterologous TCR or CAR may bind and/or bind specifically and/or bind selectively a peptide presenting molecule for example an HLA presenting or displaying a cancer and/or tumour antigen or peptide antigen thereof, i.e. a peptide fragment of a cancer and/or tumour antigen (pHLA), wherein the HLA corresponds to MHC class I (A, B, and C) which all are the HLA Class1 or specific alleles thereof or the HLA corresponds to MHC class II (DP, DM, DO, DQ, and DR) or specific alleles thereof, preferably the HLA is class 1, preferably the allele is HLA-A2 or HLA-A*02 or an HLA-A2+ or HLA-A*02 positive HLA, preferably HLA-*0201. Alternatively, the heterologous TCR or CAR may bind and/or bind specifically and/or bind selectively a cancer and/or tumour antigen or peptide antigen thereof, which is not presented or displayed by HLA.

Preferably, the heterologous TCR or CAR is not naturally expressed by the immunoresponsive cells (i.e. the TCR or CAR is exogenous or heterologous). A heterologous TCR may include αβTCR heterodimers. A heterologous TCR or CAR may be a recombinant or synthetic or artificial TCR or CAR i.e. a TCR that does not exist in nature. For example, a heterologous TCR may be engineered to increase its affinity or avidity for a specific cancer and/or tumour antigen or peptide antigen thereof (i.e. an affinity enhanced TCR or specific peptide enhanced affinity receptor (SPEAR) TCR). The affinity enhanced TCR or (SPEAR) TCR may comprise one or more mutations relative to a naturally occurring TCR, for example, one or more mutations in the hypervariable complementarity determining regions (CDRs) of the variable regions of the TCR α and β chains. These mutations may increase the affinity of the TCR for MHCs that display a peptide fragment of a tumour antigen optionally when expressed by tumour and/or cancer cells. Suitable methods of generating affinity enhanced or matured TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to tumour or cancer cells expressing the tumour antigen of the MAGE family, for example MAGE A4 or peptide antigen thereof for example the sequence GVYDGREHTV, SEQ ID NO: 18.

According to the invention the heterologous TCR may be a MAGE A4 TCR which may comprise the α chain reference amino acid sequence of SEQ ID NO: 21 or a variant thereof and the β chain reference amino acid sequence of SEQ NO: 23 or a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence. The TCR may be encoded by the α chain reference nucleotide sequence of SEQ ID NO: 22 or a variant thereof and the 13 chain reference nucleotide sequence of SEQ NO: 24 or a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence.

According to the present invention the TCR may comprise a TCR alpha chain variable domain and a TCR beta chain variable domain, wherein:
(i) the alpha chain variable domain comprises CDRs having the sequences VSPFSN (αCDR1), SEQ ID NO:27 or amino acids 48-53 of SEQ ID NO:21, LTFSEN (αCDR2), SEQ ID NO:28 or amino acids 71-76 of SEQ ID NO:21, and CVVSGGTDSWGKLQF (αCDR3), SEQ ID NO:29 or amino acids 111-125 of SEQ ID NO:21, and
(ii) the beta chain variable domain comprises CDRs having the sequences KGHDR (βCDR1), SEQ ID NO:30 or amino acids 46-50 of SEQ ID NO:23, SFDVKD (βCDR2), SEQ ID NO:31 or amino acids 68-73 of SEQ ID NO:23, and CATSGQGAYEEQFF (βCDR3), SEQ ID NO:32 or amino acids 110-123 of SEQ ID NO:23 or sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto, optionally 100% sequence identity thereto.

Accordingly, the TCR may comprise a TCR in which the alpha chain variable domain comprises an amino acid sequence that has at least 70%, 75%, 80%, 85%, 90%, 95%, 96%, 97%, 98% or 99% identity to SEQ ID NO:25 or the sequence of amino acid residues 1-136 of SEQ ID NO:22, and/or the beta chain variable domain comprising an amino acid sequence that has at least 70%, 75%, 80%, 85%, 90%, 95%, 96%, 97%, 98% or 99% identity to SEQ ID NO:26 or the sequence of amino acid residues 1-133 of SEQ ID NO:23.

The terms “progenitor TCR”, is used herein to refer to a TCR comprising the MAGE A4 TCR α chain and MAGE A4 TCR β chain of SEQ ID NOs: 21 and 23 respectively. It is desirable to provide TCRs that are mutated or modified relative to the progenitor TCR that have an equal, equivalent or higher affinity and/or an equal, equivalent or slower off-rate for the peptide-HLA complex than the progenitor TCR. According to the invention the heterologous TCR may have more than one mutation present in the alpha chain variable domain and/or the beta chain variable domain relative to the progenitor TCR and may be denoted, “engineered TCR” or “mutant TCR”. These mutation(s) may improve the binding affinity and/or specificity and/or selectivity and/or avidity for MAGE A4 or peptide antigen thereof. In certain embodiments, there are 1, 2, 3, 4, 5, 6, 7 or 8 mutations in alpha chain variable domain, for example 4 or 8 mutations, and/or 1, 2, 3, 4 or 5 mutations in the beta chain variable domain, for example 5 mutations. In some embodiments, the α chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 25. In some embodiments, the β chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 26.

According to the invention the TCR may comprise a TCR in which, the alpha chain variable domain comprises SEQ ID NO: 25 or the amino acid sequence of amino acid residues 1-136 of SEQ ID NO:21, or an amino acid sequence in which amino acid residues 1-47, 54-70, 77-110 and 126-136 thereof have at least 70%, 75%, 80%, 85%, 90%, 95%, 96%, 97%, 98% or 99% identity to the sequence of amino acid residues 1-47, 54-70, 77-110 and 126-136 respectively of SEQ ID NO:25 and/or in which amino acid residues 48-53, 71-76 and 111-125, CDR 1, CDR 2, CDR 3 respectively, have at least 70%, 75%, 80%, 85%, 90%, 95%, 96%, 97%, 98% or 99% identity to the sequence of amino acid residues 48-53, 71-76 and 111-125, CDR 1, CDR 2, CDR 3, respectively of SEQ ID NO:25.

According to the invention, the TCR may comprise a TCR in which, in the alpha chain variable domain, the sequence of:
(i) amino acid residues 1-47 thereof may have (a) at least 70%, 75%, 80%, 85%, 90% or 95% identity to the sequence of amino acid residues 1-47 of SEQ ID NO:25 or (b) may have one, two or three amino acid residues inserted or deleted relative to residues 1-47 of SEQ ID NO:25,
(ii) amino acid residues 48-53 is VSPFSN, CDR 1, SEQ ID NO:27 or amino acids 48-53 of SEQ ID NO:25,
(iii) amino acid residues 54-70 thereof may have (a) at least 70%, 75%, 80%, 85%, 90% or 95% identity to the sequence of amino acid residues 54-70 of SEQ ID NO: 25 or (b) may have one, two or three amino acid residues inserted or deleted relative to the sequence of amino acid residues 54-70 of SEQ ID NO: 25,
(iv) amino acid residues 71-76 may be LTFSEN, CDR 2, SEQ ID NO:28 or amino acids 71-76 of SEQ ID NO:25,
(v) amino acid residues 77-110 thereof may have at least 70%, 75%, 80%, 85%, 90% or 95% identity to the sequence of amino acid residues 77-110 of SEQ ID NO:25 or may have one, two or three insertions, deletions or substitutions relative to the sequence of amino acid residues 77-110 of SEQ ID NO:25,
(vi) amino acids 111-125 may be CVVSGGTDSWGKLQF, CDR 3, SEQ ID NO:29 or amino acids 111-125 of SEQ ID NO:25,
(vii) amino acid residues 126-136 thereof may have at least 70%, 75%, 80%, 85%, 90% or 95% identity to the sequence of amino acid residues 126-136 of SEQ ID NO: 25 or may have one, two or three insertions, deletions or substitutions relative to the sequence of amino acid residues 126-136 of SEQ ID NO:25.

According to the invention, the TCR may comprise a TCR in which, in the beta chain variable domain comprises the amino acid sequence of SEQ ID NO:26, or an amino acid sequence in which amino acid residues 1-45, 51-67, 74-109, 124-133 thereof have at least 70%, 75%, 80%, 85%, 90% or 95% identity to the sequence of amino acid residues 1-45, 51-67, 74-109, 124-133 respectively of SEQ ID NO:26 and in which amino acid residues 46-50, 68-73 and 110-123 have at least 70%, 75%, 80%, 85%, 90% or 95% identity to the sequence of amino acid residues 46-50, 68-73 and 110-123, CDR 1, CDR 2, CDR 3, respectively of SEQ ID NO:26.

According to the invention, the TCR may comprise a TCR in which, in the beta chain variable domain, the sequence of:
(i) amino acid residues 1-45 thereof may have (a) at least 70%, 75%, 80%, 85%, 90% or 95% identity to the sequence of amino acid residues 1-45 of SEQ ID NO:26 or (b) may have one, two or three amino acid residues inserted or deleted relative to residues 1-45 of SEQ ID NO:26,
(ii) amino acid residues 46-50 is KGHDR, CDR 1, SEQ ID NO:30 or amino acids 46-50 of SEQ ID NO:26,
(iii) amino acid residues 51-67 thereof may have (a) at least 70%, 75%, 80%, 85%, 90% or 95% identity to the sequence of amino acid residues 51-67 of SEQ ID NO:26 or (b) may have one, two or three amino acid residues inserted or deleted relative to the sequence of amino acid residues 51-67 of SEQ ID NO:26,
(iv) amino acid residues 68-73 may be SFDVKD, CDR 2, SEQ ID NO:31 or amino acids 68-73 of SEQ ID NO:26,
(v) amino acid residues 74-109 thereof may have at least 70%, 75%, 80%, 85%, 90% or 95% identity to the sequence of amino acid residues 74-109 of SEQ ID NO:26 or may have one, two or three insertions, deletions or substitutions relative to the sequence of amino acid residues 74-109 of SEQ ID NO:26;
(vi) amino acids 110-123 may be CATSGQGAYEEQFF, CDR 3, SEQ ID NO:32 or amino acids 110-123 of SEQ ID NO:26,
(vii) amino acid residues 124-133 thereof may have at least 70%, 75%, 80%, 85%, 90% or 95% identity to the sequence of amino acid residues 124-133 of SEQ ID NO:26 or may have one, two or three insertions, deletions or substitutions relative to the sequence of amino acid residues 124-133 of SEQ ID NO:26.

Amino acid and nucleotide sequence identity is generally defined with reference to the algorithm GAP (GCG Wisconsin Package™, Accelrys, San Diego Calif.). GAP uses the Needleman & Wunsch algorithm (J. Mol. Biol. (48): 444-453 (1970)) to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), generally employing default parameters.

Particular amino acid sequence variants may differ from a reference sequence by insertion, addition, substitution or deletion of 1 amino acid, 2, 3, 4, 5-10, 10-20 or 20-30 amino acids. In some embodiments, a variant sequence may comprise the reference sequence with 1, 2, 3, 4, 5, 6, 7, 8, 9, 10 or more residues inserted, deleted or substituted. For example, up to 15, up to 20, up to 30 or up to 40 residues may be inserted, deleted or substituted.

In some preferred embodiments, a variant may differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. Conservative substitutions may, for example, be between amino acids within the following groups:
(i) alanine and glycine;
(ii) glutamic acid, aspartic acid, glutamine, and asparagine
(iii) arginine and lysine;
(iv) asparagine, glutamine, glutamic acid and aspartic acid
(v) isoleucine, leucine and valine;
(vi) phenylalanine, tyrosine and tryptophan
(vii) serine, threonine, and cysteine.

The CD8α Co-Receptor

According to the present invention, the population of modified immunoresponsive cells expressing or presenting a heterologous TCR or CAR may further express or present a heterologous co-receptor. The heterologous co-receptor may be a CD8 co-receptor. The CD8 co-receptor may comprise a dimer or pair of CD8 chains which comprises a CD8-α and CD8-β chain or a CD8-α and CD8-α chain. Preferably, the CD8 co-receptor is a CD8αα co-receptor comprising a CD8-α and CD8-α chain. A CD8α co-receptor may comprise the amino acid sequence of at least 80% identity to SEQ ID NO: 19, SEQ ID NO: 19 or a variant thereof. The CD8α co-receptor may be a homodimer.

The CD8 co-receptor binds to class 1 MHCs and potentiates TCR signalling. According to the invention the CD8 co-receptor may comprise the reference amino acid sequence of SEQ ID NO: 19 or may be a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence SEQ ID NO: 19. The CD8 co-receptor may be encoded by the reference nucleotide sequence of SEQ ID NO: 20 or may be a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence SEQ ID NO: 20.

According to the invention the heterologous CD8 co-receptor may comprise a CD8 co-receptor in which, in the Ig like V-type domain comprises CDRs having the sequence;

(i) VLLSNPTSG, CDR1, SEQ ID NO: 33, or amino acids 45-53 of SEQ ID NO: 19,

(ii) YLSQNKPK, CDR2, SEQ ID NO: 34 or amino acids 72-79 of SEQ ID NO: 19,

(iii) LSNSIM, CDR3, SEQ ID NO: 35 or amino acids 80-117 of SEQ ID NO: 19,

or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto.

According to the invention the heterologous CD8 co-receptor may comprise a CD8 co-receptor which comprises or in which, in the Ig like V-type domain comprises, residues 22-135 of the amino acid sequence of SEQ ID No:19, or an amino acid sequence in which amino acid residues 22-44, 54-71, 80-117, 124-135 thereof have at least 70%, 75%, 80%, 85%, 90% or 95% identity to the sequence of amino acid residues 22-44, 54-71, 80-117, 124-135, CDR 1, CDR 2, CDR 3, respectively of SEQ ID No:19 and in which amino acid residues 45-53, 72-79 and 118-123 have at least 70%, 75%, 80%, 85%, 90% or 95% identity to the sequence of amino acid residues 45-53, 72-79 and 118-123 respectively of SEQ ID No:19.

According to the invention the CD8 co-receptor may comprise a CD8 co-receptor in which, or in which in the Ig like V-type domain, the sequence of:
(i) amino acid residues 22-44 thereof may have (a) at least 70%, 75%, 80%, 85%, 90% or 95% identity to the sequence of amino acid residues 22-44 of SEQ ID NO:19 or (b) may have one, two or three amino acid residues inserted or deleted relative to residues 22-44 of SEQ ID NO:19,
(ii) amino acid residues 45-53 is VLLSNPTSG, SEQ ID NO:33, CDR1, or amino acids 45-53 of SEQ ID NO:19,
(iii) amino acid residues 54-71 thereof may have (a) at least 70%, 75%, 80%, 85%, 90% or 95% identity to the sequence of amino acid residues 54-71 of SEQ ID NO:19 or (b) may have one, two or three amino acid residues inserted or deleted relative to the sequence of amino acid residues 54-71 of SEQ ID NO:19,
(iv) amino acid residues 72-79 may be YLSQNKPK, CDR2, SEQ ID NO:34 or amino acids 72-79 of SEQ ID NO:19,
(v) amino acid residues 80-117 thereof may have at least 70%, 75%, 80%, 85%, 90% or 95% identity to the sequence of amino acid residues 80-117 of SEQ ID NO:19 or may have one, two or three insertions, deletions or substitutions relative to the sequence of amino acid residues 80-117 of SEQ ID NO:19;
(vi) amino acids 118-123 may be LSNSIM, CDR3, SEQ ID NO:35 or amino acids 80-117 of SEQ ID NO:19,
(vii) amino acid residues 124-135 thereof may have at least 70%, 75%, 80%, 85%, 90% or 95% identity to the sequence of amino acid residues 124-135 of SEQ ID NO:19 or may have one, two or three insertions, deletions or substitutions relative to the sequence of amino acid residues 124-135 of SEQ ID NO:19.

The modified immunoresponsive cells that express heterologous CD8 co-receptor may demonstrate improved affinity and/or avidity and/or improved T-cell activation, as determinable by the assays disclosed herein, towards or on stimulation by antigenic peptide, tumour or cancer antigen optionally when presented on HLA relative to modified immunoresponsive cells that do not express heterologous CD8 co-receptor. The heterologous CD8 of modified immunoresponsive cells may interact or bind specifically to an MHC, the MHC may be class I or class II, preferably class I major histocompatibility complex (MHC), HLA-I molecule or with the MHC class I HLA-A/B2M dimer, preferably the CD8-α interacts with the a3 portion of the Class I MHC (between residues 223 and 229), preferably via the IgV-like domain of CD8. Accordingly the heterologous CD8 improves TCR binding of the immunoresponsive cells to the HLA and/or antigenic peptide bound or presented by HLA pMHCI or pHLA, optionally on the surface of antigen presenting cell, dendritic cell and/or tumour or cancer cell, tumour or cancer tissue compared to immunoresponsive cells lacking the heterologous CD8. Accordingly the heterologous CD8 can improve or increase the off-rate (k_{off}) of the cell (TCR)/peptide-major histocompatibility complex class I (pMHCI) interaction of the immunoresponsive cells, and hence its half-life, optionally on the surface of antigen presenting cell, dendritic cell and/or tumour or cancer cell, or tumour or cancer tissue compared to the cells lacking the heterologous CD8, and thereby may also provide improved ligation affinity and/or avidity. The heterologous CD8 can improve organizing the TCR on the immunoresponsive cell surface to enable cooperativity in pHLA binding and may provide improved therapeutic avidity. Accordingly, the heterologous CD8 co-receptor modified immunoresponsive cells may bind or interact with LCK (lymphocyte-specific protein tyrosine kinase) in a zinc-dependent manner leading to activation of transcription factors like NFAT, NF-κB, and AP-1.

According to the invention the modified immunoresponsive cells may have an improved or increased expression of CD40L, cytokine production, cytotoxic activity, induction of dendritic cell maturation or induction of dendritic cell cytokine production, optionally in response to cancer and/or tumour antigen or peptide antigen thereof optionally as presented by tumour of cancer cell or tissue, in comparison to immunoresponsive cells lacking the heterologous CD8 co-receptor.

Therapeutic Effects

According to the present invention and the methods and uses of the present invention, the T-cell function is enhanced by at least 10%, alternatively 20%, 30%, 40%, 50%, 60%, 70%, 80%, 90%, 100%, 120%, 150%, 200% or more relative to such levels before the treatment or intervention or relative to treatment with either a PD-1 axis binding antagonist alone or modified immunoresponsive cells expressing or presenting a heterologous TCR alone, for example as judged by increased secretion of y-interferon from CD8+ T-cells, increased T-cell proliferation, increased internal signalling, increased antigen responsiveness, increased secretion of cytokines and/or interferon, increased target cell killing, increased T-cell activation, increased CD28 signalling, increased T-cell ability to infiltrate tumour, increased ability to recognise and bind to dendritic cell presented antigen.

According to the present invention and the methods and uses of the present invention, tumour immunity or evasion of immune recognition by the tumour may be attenuated resulting in improved tumour recognition and attack by the immune system and thereby treating tumour immunity for example as measured by tumour binding, tumour shrinkage and tumour clearance. Accordingly, the present invention provides treatment of tumour immunity and/or provides treatment of tumour immunity which is enhanced by at least 10%, alternatively 20%, 30%, 40%, 50%, 60%, 70%, 80%, 90%, 100%, 120%, 150%, 200% or more relative to such levels before the treatment or intervention or relative to treatment with either a PD-1 axis binding antagonist alone or modified immunoresponsive cells expressing or presenting a heterologous TCR alone, for example as measured by any one or more of tumour binding, tumour shrinkage and tumour clearance.

According to the present invention and the methods and uses of the present invention there is provided improved or enhanced tumour immunogenicity, for example as measured by the ability to provoke an immune response in response to tumour or tumour antigen, for example enhanced by at least 10%, alternatively 20%, 30%, 40%, 50%, 60%, 70%, 80%, 90%, 100%, 120%, 150%, 200% or more relative to such levels before the treatment or intervention or relative to treatment with either a PD-1 axis binding antagonist alone or modified immunoresponsive cells expressing or presenting a heterologous TCR alone, for example as judged by any one or more of increased secretion of cytokines and/or interferon, increased T-cell proliferation, increased antigen responsiveness, target cell killing, T-cell activation, CD28 signalling, T-cell ability to infiltrate tumour, ability to recognise and bind to dendritic cell presented antigen.

According to the present invention and the methods and uses of the present invention there is provided an improved sustained response of reducing tumour growth or tumour growth rate or maintaining tumour size after cessation of treatment, for example, as determined by any one or more of the measurement of tumour size or tumour number preferably enhanced by at least 10%, alternatively 20%, 30%, 40%, 50%, 60%, 70%, 80%, 90%, 100%, 120%, 150%, 200% or more relative to such levels before the treatment or intervention or relative to treatment with either a PD-1 axis binding antagonist alone or modified immunoresponsive cells expressing or presenting a heterologous TCR alone. For example the sustained response may have a duration at least the same as the treatment duration, at least 1.5, 2.0, 2.5, or 3.0 or more times the length of the treatment duration.

In the context of T-cell activity the term “dysfunction” refers to a state of reduced immune responsiveness to antigenic stimulation and includes T-cell exhaustion and/or anergy whereby the T-cell may recognise and bind antigen but shows reduced effectiveness in progressing immune response or combating tumour growth. Dysfunctional T-cells demonstrate impaired capacity to translate antigen recognition into down-stream T-cell effector functions, such as proliferation, cytokine and interferon production or target cell killing and/or appear refractory or unresponsive to antigen recognition as is characteristic of T-cell dysfunctional disorder. “T-cell dysfunctional disorder” may be associated with inappropriate increased T-cell signalling through PD-1; T-cells having decreased ability to proliferate and/or produce cytokines and/or cytolytic activity; T-cell anergy; tumour immunity.

“T-cell exhaustion” comprises a state of T cell dysfunction due to sustained TCR signalling as part of the response to cancer and prevents optimal response to tumours. Exhaustion can find effect through either the cell intrinsic negative regulatory (costimulatory) pathways (for example PD-1, PD-1 axis, B7-H3, B7-H4) or through the cell extrinsic negative regulatory pathways (immunoregulatory cytokines). T-cell exhaustion is characterised by poor effector function, sustained expression of inhibitory receptors and an altered activity of transcription distinct from that of functional effector or memory T-cells. T-cell anergy occurs through deficient signalling through the T-cell receptor and a resulting state of unresponsiveness to antigen stimulation often even in the context of costimulation, consequently such T-cells do not undergo clonal expansion and/or acquire effector functions.

Administration

According to the invention the PD-1 axis binding antagonist and modified immunoresponsive cells can be administered separately, sequentially or simultaneously.

Accordingly,

(a) the PD-1 axis binding antagonist may be administered prior to, simultaneous with or after the modified immunoresponsive cells, or

(b) the PD-1 axis binding antagonist may be administered prior to and simultaneously with the modified immunoresponsive cells, or

(c) the PD-1 axis binding antagonist may be administered prior to and after the modified immunoresponsive cells, or

(d) the PD-1 axis binding antagonist may be administered simultaneously with and after the modified immunoresponsive cells, or

(e) the PD-1 axis binding antagonist may be administered after the modified immunoresponsive cells, or

(f) the PD-1 axis binding antagonist may be administered prior to and simultaneously with and after the modified immunoresponsive cells.

For example, the first administration of the PD-1 axis binding antagonist after the administration of the modified immunoresponsive cells may be at any one of days 14, 15, 16, 17, 18, 19, 20, 21, 22, 23, 24, 25, 26, 27, 28, 29, 30, 31, 32, 33, 34, or 35 after the modified immunoresponsive cells are administered, preferably days 14 to 16 or 17 to 21 or 22 to 26, preferably days 17 to 22, preferably either day 17, 21 or 22.

According to the invention the PD-1 axis binding antagonist and/or the modified immunoresponsive cells may be administered continuously, optionally as a single dose or intermittently, optionally as more than one dose.

According to the invention the modified immunoresponsive cells may be administered as a single dose. The modified immunoresponsive cells may be administered at a dose of between about 500 million to any one of about 1 billion cells, about 2 billion cells, about 3 billion cells, about 4 billion cells, about 5 billion cells, about 6 billion cells, about 7 billion cells, about 8 billion cells, about 9 billion cells, about 10 billion cells, about 11 billion cells, about 12 billion cells, about 13 billion cells, about 14 billion cells, about 15 billion cells, about 16 billion cells, about 17 billion cells, about 18 billion cells, about 19 billion cells, about 20 billion cells, or about 21 billion cells. The modified immunoresponsive cells may be administered at a dose of between about 100 million to about 200 million cells, about 300 million to about 400 million cells, about 500 million to about 600 million cells, about 700 million to about 800 million cells, or about 900 million to about 1 billion cells, optionally about 500 million to about 1 billion cells, about 2 billion to about 5 billion cells or about 6 billion to about 10 billion cells.

According to the invention the PD-1 axis binding antagonist and/or the modified immunoresponsive cells may be administered, intravenously, intramuscularly, subcutaneously, topically, orally, transdermally, intraperitoneally, intraorbitally, by implantation, by inhalation, intrathecally, intraventricularly, or intranasally or by intravenous infusion. Preferably, the PD-1 axis binding antagonist and/or the modified immunoresponsive cells may be administered intravenously or by intravenous infusion.

According to the invention the PD-1 axis binding antagonist may be administered at a dose of any of about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg/kg. The PD-1 axis binding antagonist can be administered at a dose of any of about 0.5 mg/kg to about 1.5 mg/kg, about 1 mg/kg to about 2 mg/kg, about 3 mg/kg to about 5 mg/kg, about 6 mg/kg to about 9 mg/kg, about 10 mg/kg to about 15 mg/kg, about 16 mg/kg to about 20 mg/kg, about 20 mg/kg to about 25 mg/kg, about 1 mg/kg to about 9 mg/kg, about 10 mg/kg to about 20 mg/kg. According to the invention the PD-1 axis binding antagonist may be administered at a dose of any of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900, 950, 1000 mg, preferably 200, 250 or 300 mg. According to the invention the PD-1 axis binding antagonist may be administered at a fixed dose, alternatively the dose may be varied, in either case for example where there is more than one dose or in a dosing cycle.

According to the invention the PD-1 axis binding antagonist can be administered as

(a) a single dose in each of one or more dosing cycles,

(b) one or more doses in each of one or more dosing cycles,

(c) a single dose on the first day of each of one or more dosing cycles,

(d) one or more doses in each of one or more dosing cycles comprising a dose on the first day of each of the one or more dosing cycles,

(e) one or more doses in each of one or more dosing cycles, at least one dose being on the first day of each cycle.

According to the invention the PD-1 axis binding antagonist can be administered in a dosing cycle wherein the dosing cycle can be any of 5, 6, 7, 8, 9, 10, 11, 12, 13, 14, 15, 16, 17, 18, 19, 20, 21, 22, 23, 24, 25, 26, 27, 28, 29, 30, 31 days, 1 month, 2 months, 3 months, 4 months, 5 months, 6 months, 7 months, 8 months, 9 months, 10 months, 11 months, or 12 months. Accordingly the dosing cycle can be any of 10 to 12 days, 11 to 13 days, 14 to 17 days, 14 to 17 days, 18 to 21 days, 22 to 24 days, 24 to 27 days, 28 to 30 days or 31 days, one week, two weeks, three weeks, four weeks or one month, 2 months 6 months, preferably 3 weeks or 21 days.

According to the invention the PD-1 axis binding antagonist can administered as one or more doses on each of one or more dosing cycles, prior to administration of the modified immunoresponsive cells and administered as one or more doses on each of one or more dosing cycles after administration of the modified immunoresponsive cells, optionally wherein at least one dose can be on the first day of each cycle. Alternatively the PD-1 axis binding antagonist can administered as one or more doses on each of one or more dosing cycles, prior to administration of the modified immunoresponsive cells, administered simultaneously with administration of the modified immunoresponsive cells (optionally as a single dose) and administered as one or more doses on each of one or more dosing cycles after administration of the modified immunoresponsive cells, optionally wherein at least one dose can be on the first day of each cycle. Alternatively, the PD-1 axis binding antagonist can be administered as one or more doses on each of one or more dosing cycles after administration of the modified immunoresponsive cells, optionally wherein at least one dose can be on the first day of each cycle.

According to the invention the PD-1 axis binding antagonist can administered as a single dose on each of one or more dosing cycles, prior to administration of the modified immunoresponsive cells and administered as a single dose on each of one or more dosing cycles after administration of the modified immunoresponsive cells, optionally wherein the single dose can be on the first day of each cycle. Alternatively the PD-1 axis binding antagonist can administered as a single dose on each of one or more dosing cycles, prior to administration of the modified immunoresponsive cells, administered simultaneously with administration of the modified immunoresponsive cells (optionally wherein the modified immunoresponsive cells are administered as a single dose) and administered as a single dose on each of one or more dosing cycles after administration of the modified immunoresponsive cells, optionally wherein the single dose can be on the first day of each cycle. Alternatively the PD-1 axis binding antagonist can administered as a single dose on each of one or more dosing cycles, after the administration of the modified immunoresponsive cells (optionally wherein the modified immunoresponsive cells are administered as a single dose), optionally wherein the single dose can be on the first day of each cycle. According to the invention the PD-1 axis binding antagonist can be administered as a single fixed dose of about 200 mgs on the first day of each of one or more dosing cycles, wherein the dosing cycle is 21 days and/or wherein the modified immunoresponsive cells are administered as a single dose of between about 5 billion and about 10 billion cells, optionally wherein the first dose of PD-1 axis binding antagonist after the modified immunoresponsive cells are administered is on either day 17, day 21 or day 22 after the immunoresponsive cells are administered.

According to the invention the PD-1 axis binding antagonist can administered for a specified period, meaning also that the PD-1 axis binding antagonist dosing cycles can administered for a specified period, for example a specific period after administration of the modified immunoresponsive cells. The specified period may be any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 months, preferably 24 months.

The method according to the invention may comprise the steps wherein

(a) the PD-1 axis binding antagonist is administered as one or more doses on each of one or more dosing cycles prior to administration of the modified immunoresponsive cells, optionally wherein at least one dose can be on the first day of each cycle,

(b) the status of disease is determined at a point after the PD-1 axis binding antagonist administration but prior to administration of the modified immunoresponsive cells and compared to the status prior to PD-1 axis binding antagonist administration, wherein if stable disease or progressive disease is determined then,

(c) modified immunoresponsive cells are administered and the PD-1 axis binding antagonist is administered as one or more doses on each of one or more dosing cycles after the administration of the modified immunoresponsive cells or simultaneously with and after the administration of the modified immunoresponsive cells; optionally wherein at least one dose can be on the first day of each cycle, further optionally the PD-1 axis binding antagonist can be administered for a specified period. Further optionally, the first dose of PD-1 axis binding antagonist after the modified immunoresponsive cells are administered is on either day 17, day 21 or day 22 after the immunoresponsive cells are administered.

The method according to the invention may comprise the steps wherein

(a) the PD-1 axis binding antagonist is administered as one or more doses on each of one or more dosing cycles prior to the administration of the modified immunoresponsive cells, optionally wherein at least one dose can be on the first day of each cycle,

(b) the status of disease is determined at the point after the PD-1 axis binding antagonist administration but prior to administration of the modified immunoresponsive cells and compared to the status prior to PD-1 axis binding antagonist administration, wherein if complete response or partial response is determined then,

(c) the PD-1 axis binding antagonist is administered as one or more doses on each of one or more dosing cycles without administration of the modified immunoresponsive cells; optionally wherein at least one dose can be on the first day of each cycle, further optionally wherein the PD-1 axis binding antagonist is administered for the shorter of a specified period or determination of disease progression. Further optionally where in step (c) stable disease or progressive disease is determined then, (d) modified immunoresponsive cells are administered and the PD-1 axis binding antagonist is administered as one or more doses on each of one or more dosing cycles after the administration of the modified immunoresponsive cells or simultaneously with and after the administration of the modified immunoresponsive cells; optionally wherein at least one dose can be on the first day of each cycle, further optionally the PD-1 axis binding antagonist can be administered for a specified period. Further optionally the first dose of PD-1 axis binding antagonist after the modified immunoresponsive cells are administered is on either day 17, day 21 or day 22 after the immunoresponsive cells are administered.

According to the invention a “complete response” (CR) is determined where all target lesions or tumours have been assessed or measured as having disappeared. “Partial response” (PR) is determined when there is a measurement of an at least a 30% decrease in the sum of the longest diameters (SLD) of target lesions or tumours, for example as referenced to the control or pre-treatment comparator. “Progressive disease” (PD) is determined when there is a measurement of at least a 20% increase in the sum of the longest diameters (SLD) of target lesions or tumours, for example as referenced to the control or pre-treatment comparator, since the treatment started or the presence of one or more new lesions. “Stable disease” (SD) is determined where it is determined that there is neither sufficient reduction or decrease in the sum of the longest diameters (SLD) of target lesions or tumours to qualify for PR, nor sufficient increase to qualify for PD, taking as reference the smallest SLD since the treatment started.

According to the present invention the cancer can be relapsed cancer or refractory cancer or recurrent cancer or locally recurrent cancer or metastatic cancer, non-resectable cancer or locally confined, cancer with no surgical or radiotherapy option or inoperable cancer, or any combination thereof. The subject may have relapsed cancer or refractory cancer or recurrent cancer or locally recurrent cancer or metastatic cancer or locally confined or inoperable cancer, or any combination thereof.

According to the present invention the cancer and/or tumour may express a MAGE protein, peptide, antigen or peptide antigen thereof and/or express PD-L1 or PD-L2 [optionally with CPS≥1], optionally MAGE-A4 protein, peptide, antigen or peptide antigen thereof and/or express PD-L1 or PD-L2 [optionally with CPS≥1].

According to the present invention the cancer may be selected from; lung cancer, non-small cell lung cancer (NSCLC), metastatic or advanced NSCLC, squamous NSCLC, adenocarcinoma NSCLC, adenosquamous NSCLC, large cell NSCLC, ovarian cancer, gastric cancer, urothelial cancer, esophageal cancer, esophagogastric junction cancer (EGJ), melanoma, bladder cancer, head and neck cancer, head and neck squamous cell carcinoma (HNSCC), cancer of the oral cavity, cancer of the oropharynx, cancer of the hypopharynx, cancer of the throat, cancer of the larynx, cancer of the the tonsil, cancer of the tongue, cancer of the soft palate, cancer of the pharynx, synovial sarcoma, myxoid round cell liposarcoma (MRCLS), optionally wherein the cancer or tumour express a MAGE protein, peptide, antigen or peptide antigen thereof and/or express PD-L1 or PD-L2 [optionally with CPS≥1], optionally MAGE-A4 protein, peptide, antigen or peptide antigen thereof and/or express PD-L1 or PD-L2 [optionally with CPS≥1], CPS meaning combined positive score.

According to the present invention the cancer may be selected from any one of breast cancer, metastatic breast cancer, liver cancer, renal cell carcinoma, synovial sarcoma, urothelial cancer or tumour, pancreatic cancer, colorectal cancer, metastatic stomach cancer, metastatic gastric cancer, metastatic liver cancer, metastatic ovarian cancer, metastatic pancreatic cancer, metastatic colorectal cancer, metastatic lung cancer, colorectal carcinoma or adenocarcinoma, lung carcinoma or adenocarcinoma, pancreatic carcinoma or adenocarcinoma, mucinous adenoma, ductal carcinoma of the pancreas, hematological malignancy, optionally wherein the cancer or tumour express a MAGE protein, peptide, antigen or peptide antigen thereof and/or express PD-L1 or PD-L2 [optionally with CPS≥1], optionally MAGE-A4 protein, peptide, antigen or peptide antigen thereof and/or express PD-L1 or PD-L2 [optionally with CPS≥1]. According to the invention the cancer may be recurrent or metastatic HNSCC with disease progression on after platinum containing chemotherapy.

There is further provided the method or use according to the invention wherein the subject has not received prior cancer treatment, alternatively wherein the subject has received prior cancer treatment and/or has failed to respond to prior cancer treatment or wherein the disease has progressed during or following prior treatment.

According to the invention the prior treatment can comprise systemic and/or local therapy, for example any one or more of; surgery, radiation therapy, cryotherapy, laser therapy, topical therapy, chemotherapy, hormonal therapy, targeted drugs, or immunotherapy. Accordingly, the prior treatment can comprise local therapy, for example any one or more of surgery, radiation therapy cryotherapy, laser therapy, topical therapy and/or systemic therapy, for example any one or more of chemotherapy, hormonal therapy, targeted drugs, or immunotherapy.

According to the invention the prior treatment can comprise a PD-1 axis binding antagonist, PD-L1 binding antagonist or PD-1 binding antagonist. Accordingly the prior treatment can comprise any of;

(a) an anti-PD-L1 antibody which inhibits binding between PD-L1 and PD-1 and/or between PD-L1 and B7-1,

(b) an anti-PD-L1 antibody which inhibits PD-L1 on the cancer cell surface from transducing a signal to the intracellular pathway,

(c) an anti-PD-1 antibody which inhibits binding between PD-L1 and PD-1 and/or between PD-L2 and PD-1,

(d) an anti-PD-1 antibody which inhibits PD-1 on the T cell surface from transducing a signal to the intracellular pathway,

(e) a PD-L1 binding antagonist which is selected from;
(i) Durvalumab, Imfinzi or MEDI4736,
(ii) Atezolizumab, Tecentriq or MPDL3280A,
(iii) Avelumab, Bavencio or MSB0010718C,
(iv) MDX-1105, BMS-936559,

(f) a PD-1 binding antagonist is selected from;
(i) Pembrolizumab, Keytruda, Lambrolizumab or MK-3475,
(ii) Cemiplimab, Libtayo, or REGN-2810,
(iii) BMS/ONO, Nivolumab, Opdivo, ONO-4538, BMS-936558 or MDX1106.

According to the invention the prior treatment may comprise an Epidermal Growth Factor Receptor Antagonist, optionally Cetuximab According to the invention when the prior treatment comprises chemotherapy this may comprise one or more platinum compound, optionally selected from Lipoplatin, Cisplatin, Carboplatin, Oxaliplatin, Nedaplatin, Triplatin tetranitrate, Phenanthriplatin, Satraplatin, Picoplatin. Additionally or alternatively when the prior treatment comprises chemotherapy this may comprise one or more chemotherapeutic agent selected from, methotrexate, capecitabine, taxane, anthracycline, paclitaxel, docetaxel, paclitaxel protein bound particles, doxorubicine, epirubicine, 5-fluorouracil, cyclophosphamide, afatinib, vincristine, etoposide or combinations thereof. Additionally or alternatively when the prior treatment comprises chemotherapy this may comprise one or more chemotherapeutic agent selected from, FEC: 5-fluorouracil, epirubicine, cyclophosphamide; FAC: 5-fluorouracil, doxorubicine, cyclophosphamide; AC: doxorubicine, cyclophosphamide; EC: epirubicine, cyclophosphamide

According to the invention the subject may not have received prior treatment in recurrence less than or equal to 12 months since the last treatment or less than or equal to 6 months since the last treatment.

According to the invention the subject may be PD-1 axis binding antagonist, for example Pembrolizumab, Cemiplimab, Nivolumab or Opdivo, naïve, or may have received 1, 2, 3, 4, 5, 6, or 7 prior doses of PD-1 axis binding antagonist before treatment. According to the invention the subject may have received 1, 2 or 3 prior line of platinum therapy before treatment or have had a maximum of 1 prior line of platinum therapy before treatment.

According to the invention the subject may have not received any prior adjuvant therapy (surgery followed by radiation and/or chemotherapy) in recurrence less than or equal to 12 months since the last treatment or in recurrence less than or equal to 6 months since the last treatment.

According to the invention the treatment extends or improves or effectively extends or effectively improves any one or more of:

(a) progression free survival,

(b) time to progression,

(c) duration of response,

(d) overall survival,

(e) objective response or objective response rate,

(f) overall response or overall response rate,

(g) partial response or partial response rate,

(h) complete response or complete response rate;

(i) stable disease rate or median stable disease

(j) median progression free survival,

(k) median time to progression,

(l) median duration of response, or

(m) median overall survival;

(n) median objective response or median objective response rate,

(o) median overall response or median overall response rate,

(p) median partial response or median partial response rate,

(q) median complete response or median complete response,

(r) median stable disease rate or median stable disease,

in comparison to treatment with the or a PD-1 axis binding antagonist alone or modified immunoresponsive cells alone.

According to the invention the treatment extends or improves or effectively extends or effectively improves any one or more of:

(a) Best Overall Response (BOR), (b) Time to Confirmed Response (TTR), (c) Duration of Response (DoR), (d) Duration of Stable Disease (DoSD), (e) Progression Free Survival (PFS), or (f) Overall Survival (OS); in comparison to treatment with the or a PD-1 axis binding antagonist alone or modified immunoresponsive cells alone.

Best Overall Response (BOR), can be defined as the best response recorded from the date of T cell infusion until disease progression. Time to Confirmed Response (TTR), can be defined as the duration between T cell infusion and the initial date of the confirmed response. Duration of Response (DoR), can be defined as the duration from the initial date of the confirmed response to the date of PD, progressive disease (or death). Duration of Stable Disease (DoSD), can be defined as the duration from the date of T cell infusion to the date of PD, progressive disease (or death). Progression Free Survival (PFS), can be defined as the interval between the date T cell infusion and the earliest date of disease progression based on RECIST v1.1 or death due to any cause. Overall Survival (OS), can be defined the duration between T cell infusion and death due to any cause.

“Progression free survival” (PFS) refers to the time from treatment (or randomization) to first disease progression or death. “Time to progression” (TTP) does not count patients who die from causes other than the cancer or tumour being treated but is otherwise equivalent to PFS. “Duration of response” (DoR), is the length of time that cancer, tumour or lesion continues to respond to treatment without growing or spreading. According to the invention DoR, TTP and PFS can be assessed by Response Evaluation Criteria in Solid Tumors (RECIST) or can be assessed by CA-125 levels as a determinant of progression.

According to the invention PFS and/or TTP and/or DoR, or median thereof, can be or can be extended or improved by, at least 2 weeks, 3 weeks, 1 month, 2 months, 2.3 months, 2.5 months, 2.9 months, 3 months, 3.5 months, 3.8 months, 4 months, 4.5 months, 5 months, 6 months, 7 months, 8 months, 9 months, 10 months, 11 months, 12 months, 16 months, 18 months, 20 months, 22 months, 2 years, 3 years, 4 years, 5, years, 6 years, 7 years, 8 years, 9 years, 10 years, for example in comparison to treatment with PD-1 axis binding antagonist alone (control) or modified immunoresponsive cells alone (control). In one embodiment, the PFS and/or TTP and/or DoR, or median thereof, is extended about 2.9 months to 3.8 months compared to the control. In one embodiment, the PFS and/or TTP and/or DoR, or medians thereof, is extended at least about 3.8 months compared to the control. In another embodiment, the PFS and/or TTP and/or DoR, or median thereof, is extended by about 2.3 months, in one embodiment, the PFS and/or TTP and/or DoR, or median thereof, is extended about 6 months compared to a control.

“Overall survival” refers to a subject remaining alive for a defined period of time. According to the invention the overall survival, or median thereof, can be, or can be improved or extended by about 6 months, about 1 year, about 1.5 years, about 2 years, about 3 years, about 4 years, about 5 years, about 6 years, about 7 years, about 8 years, about 9 years, about 10 years, from initiation of the method or treatment according to the invention or from initial diagnosis, optionally the event used for survival analysis can be death from any cause. “Survival” refers to a subject remaining alive and includes progression free survival (PFS) and overall survival (OS). “Overall survival” is the length of time from either the date of diagnosis or the start of treatment for the disease, tumour and/or cancer, that subjects diagnosed with the disease are still alive. Survival can be estimated by the Kaplan-Meier method, and any differences in survival are computed using the stratified log-rank test; “extending survival” or “increasing the likelihood of survival” is meant increasing PFS and/or OS in a treated subject in comparison to treatment with the PD-1 axis binding antagonist alone (control) or modified immunoresponsive cells alone (control). According to the invention overall survival or survival can be, or can be extended or improved by at least 1 month, 2 months, 2.3 months, 2.5 months, 2.9 months, 3 months, 3.5 months, 3.8 months, 4 months, 4.5 months, 5 months, 6 months, 7 months, 8 months, 9 months, 10 months, 11 months, 12 months, 16 months, 18 months, 20 months, 22 months, 2 years, 3 years, 4 years, 5, years, 6 years, 7 years, 8 years, 9 years, 10 years, for example in comparison to treatment with PD-1 axis binding antagonist alone (control) or modified immunoresponsive cells alone (control).

“Objective response rate” (ObRR) is the proportion of subjects with tumour size reduction of a predefined amount, optionally determined by sum of the longest diameters (SLD) of target lesions or tumours, and for a minimum time period. “Overall response rate (ORR)” is defined as the proportion of subjects who have a partial or complete response to therapy; it does not include stable disease. ORR is generally defined as the sum of complete responses (CR) and partial responses (PRs) over a specified time period. According to the invention ObRR and/or ORR and/or PR and/or CR and/or SD can be, or can be extended or improved by at least 5%, 10%, 15%, 20%, 25%, 30%, 35%, 40%, 45%, 50%, 55%, 60%, 65%, 70%, 75%, 80%, 85%, 90%, 95%, for example in comparison to treatment with PD-1 axis binding antagonist alone (control) or modified immunoresponsive cells alone (control).

According to the present invention, the method may further comprise determining the expression level of a biomarker in a sample from the subject wherein the level of the biomarker is compared to a reference level in order to determine the subject's likelihood to respond to the treatment or to determine the subject's level of response to the treatment, wherein the sample is obtained either before during or after the treatment. The reference level may be the level prior to treatment of the subject or may be the level associated with the presence of cancer or the lack of presence of cancer. The biomarker may be a T-effector-associated gene, for example CD8A, perforin (PRF1), granzyme A (GZMA), granzyme B (GZMB), interferon-γ (IFN-v), CXCL9, or CXCL10. The biomarker may be an activated stroma-associated gene, for example transforming growth factor-β (TGF-β), fibroblast-activated protein (FAP), podplanin (PDPN), a collagen gene, or biglycan (BGN). The biomarker may be a or a myelokJ-derived suppressor cell-associated gene, for example CD68, CD163, FOXP3, or androgen-regulated gene 1. Alternatively the biomarker may be PD-L1, CD8, or androgen receptor (AR) gene.

The invention further provides a method of enhancing immune function in a subject having cancer and/or tumour comprising administering to the subject a treatment regimen comprising an effective amount of a PD-1 axis binding antagonist and a population of modified immunoresponsive cells expressing or presenting a heterologous TCR, for example as herein before described with reference to the method of treatment and the aspects and embodiments and features relating thereto.

Accordingly, the invention also provides a method of enhancing immune function wherein:

(a) CD8 T cells in the individual have enhanced priming, activation, proliferation and/or cytolytic activity relative to prior to the administration of the treatment or relative to treatment with PD-1 axis binding antagonist alone or modified immunoresponsive cells alone,

(b) the number of CD8 T cells is elevated relative to prior to administration of the treatment or relative to treatment with PD-1 axis binding antagonist alone or modified immunoresponsive cells alone,

(c) the cancer and/or tumour cells in the subject selectively have elevated expression of MHC class I antigen expression relative to prior to administration of the treatment or relative to treatment with PD-1 axis binding antagonist alone or modified immunoresponsive cells alone, optionally wherein PBMC cells of the subject do not have elevated expression of MHC class I antigen,

(d) the antigen presenting cells in the subject have enhanced maturation and activation relative to prior to administration of the treatment or relative to treatment with PD-1 axis binding antagonist alone or modified immunoresponsive cells alone, optionally wherein the antigen presenting cells are dendritic cells,

(e) the serum levels of IL-10 and/or IL-8 in the individual are reduced relative to prior to administration of the treatment or relative to treatment with PD-1 axis binding antagonist alone or modified immunoresponsive cells alone,

(f) the cancer and/or tumour of the subject has elevated levels of T-cell infiltration relative to prior to administration of the treatment or relative to treatment with PD-1 axis binding antagonist alone or modified immunoresponsive cells alone,

(g) the T cells of the subject have reduced levels of T-cell PD-1 expression relative to prior to administration of the treatment or relative to treatment with PD-1 axis binding antagonist alone or modified immunoresponsive cells alone.

Accordingly (a) the CD8 T cell activation may be characterised by an elevated frequency of gamma-IFN⁺ CD8 T cells and/or enhanced cytolytic activity relative to prior to administration of the treatment or relative to treatment with PD-1 axis binding antagonist alone or modified immunoresponsive cells alone; (b) the maturation of the antigen presenting cells may be characterised by increased frequency of CD83⁺ dendritic cells; (c) the activation of the antigen presenting cells may be characterised by elevated expression of CD80 and CD86 on dendritic cells; (d) the CD8 T cell may be an antigen-specific CD8 T cell.

According to the invention there is provided;

(a) a kit comprising a PD-1 axis binding antagonist and a package insert comprising instructions for using the PD-1 axis binding antagonist in combination with a population of modified immunoresponsive cells expressing or presenting a heterologous TCR to treat or delay the progression of cancer and/or tumour in a subject,

(b) a kit comprising a PD-1 axis binding antagonist and a population of modified immunoresponsive cells expressing or presenting a heterologous TCR, and a package insert comprising instructions for using the PD-1 axis binding antagonist and the population of modified immunoresponsive cells expressing or presenting a heterologous TCR to treat or delay the progression of cancer and/or tumour in a subject,

(c) a kit comprising a population of modified immunoresponsive cells expressing or presenting a heterologous TCR and a package insert comprising instructions for using the population of modified immunoresponsive cells expressing or presenting a heterologous TCR in combination with a PD-1 axis binding antagonist to treat or delay the progression of cancer and/or tumour in a subject;
optionally wherein the PD-1 axis binding antagonist is an anti-PD-L1 antibody, an anti-PD-1 antibody, an anti-PD-1 immunoadhesin.

According to the invention the subject, i.e. subject cancer and/or tumour, may be PD-L1 expression positive (PD-L1+) and/or HLA-A2 expression positive HLA-A2+, and/or MAGE-A4 expression positive (MAGE-A4+). According to the invention the subject (a) is not HLA-A*02:05 positive in either allele, (b) is not HLA-A*02:07 (and alleles having the same protein sequence in the antigen binding domains as A*02:07) or any A*02 null allele (designated with an “N” suffix, e.g. A*02:32N) as the sole HLA-A*02 allele (e.g. a subject with HLA alleles A*02:04 and A*02:07 is eligible), (c) does not have CNS metastases.

The invention will be further described by reference to the following figures and examples.

### FIGURES

FIG. 1. Increase in CD3 and PD-L1 detected in the post-infusion tumour of a responder

FIG. 2. increase in CD3 and PD-L1 post infusion in a non-responder

FIG. 3. MAGE-A4 SPEAR T-cells upregulate PD-1 expression upon stimulation in comparison to non-transduced (NTD) T-cells.

FIG. 4. MAGE-A4+CD8 SPEAR T-cells upregulate PD-1 expression upon stimulation in comparison to non-transduced (NTD) T-cells.

FIG. 5. PD-L1 is expressed and also upregulated by IFN-γ in A375 cells (A375.GFP MAGE-A4⁺ melanoma cell line)

FIG. 6. Scheme of pre-activation assay to stimulate and upregulate PD-1 expression on SPEAR T-cells (heterologous MAGE-A4 TCR or heterologous MAGE-A4 TCR+heterologous CD8 expressing T-cells).

FIG. 7. PD-1 is upregulated on A2M4 SPEAR T-cells by pre-activation.

FIG. 8. Supernatants of A2M4 cultures during (A) initial stimulation and (B) during re-stimulation with or without anti-PD-1 antibody. IFN-gamma ELISA data is shown here (n=6 small-scale donors).

FIG. 9. Scheme of combination A2M4 SPEAR T-cell+PD-1 axis binding antagonist (Pembrolizumab) treatment of cancer subject.

Sequences
PD1 - Human Programmed cell death protein (Homo sapiens)
SEQ ID NO: 1
MQIPQAPWPVVWAVLQLGWRPGWFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGT
YLCGAISLAPKAQIKESLRAELRVTERRAEVPTAHPSPSPRPAGQFQTLVVGVVGGLLGS
LVLLVWVLAVICSRAARGTIGARRTGQPLKEDPSAVPVFSVDYGELDFQWREKTPEPPVP
CVPEQTEYATIVFPSGMGTSSPARRGSADGPRSAQPLRPEDGHCSWPL,
PD1L1 - Human Programmed cell death 1 ligand 1(Homo sapiens)
SEQ ID NO: 2
MRIFAVFIFMTYWHLLNAFTVTVPKDLYVVEYGSNMTIECKFPVEKQLDLAALIVYWEME
DKNIIQFVHGEEDLKVQHSSYRQRARLLKDQLSLGNAALQITDVKLQDAGVYRCMISYGG
ADYKRITVKVNAPYNKINQRILVVDPVTSEHELTCQAEGYPKAEVIWTSSDHQVLSGKTT
TTNSKREEKLFNVTSTLRINTTTNEIFYCTFRRLDPEENHTAELVIPELPLAHPPNERTH
LVILGAILLCLGVALTFIFRLRKGRMMDVKKCGIQDTNSKKQSDTHLEET,
PD1L2 - Human Programmed cell death 1 ligand 2 (Homo sapiens)
SEQ ID NO: 3
MIFLLLMLSLELQLHQIAALFTVTVPKELYIIEHGSNVTLECNFDTGSHVNLGAITASLQ
KVENDTSPHRERATLLEEQLPLGKASFHIPQVQVRDEGQYQCIIIYGVAWDYKYLTLKVK
ASYRKINTHILKVPETDEVELTCQATGYPLAEVSWPNVSVPANTSHSRTPEGLYQVTSVL
RLKPPPGRNFSCVFWNTHVRELTLASIDLQSQMEPRTHPTWLLHIFIPFCIIAFIFIATV
IALRKQLCQKLYSSKDTTKRPVTTTKREVNSAI,
Durvalumab Heavy Chain Sequence
SEQ ID NO: 4
EVQLVESGGGLVQPGGSLRLSCAASGFTFSRYWMSWVRQAPGKGLEWVANIKQDGSEKYY
VDSVKGRFTISRDNAKNSLYLQMNSLRAEDTAVYYCAREGGWFGELAFDYWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCPPCPAPEFEG
GPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREPQVYTLPPSRE
EMTKNQVSLTCLVKGFYPSDIAVEWESNGQPENNYKTTPPVLDSDGSFFLYSKLTVDKSR
WQQGNVFSCSVMHEALHNHYTQKSLSLSPGK,
Durvalumab Light Chain Sequence
SEQ ID NO: 5
EIVLTQSPGTLSLSPGERATLSCRASQRVSSSYLAWYQQKPGQAPRLLIYDASSRATGIP
DRFSGSGSGTDFTLTISRLEPEDFAVYYCQQYGSLPWTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC,
Atezolizumab Heavy Chain Sequence
SEQ ID NO: 6
EVQLVESGGGLVQPGGSLRLSCAASGFTFSDSWIHWVRQAPGKGLEWVAWISPYGGSTYY
ADSVKGRFTISADTSKNTAYLQMNSLRAEDTAVYYCARRHWPGGFDYWGQGTLVTVSSAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSKLTVDKSRWQQ
GNVFSCSVMHEALHNHYTQKSLSLSPGK,
Atezolizumab Light Chain Sequence
SEQ ID NO: 7
DIQMTQSPSSLSASVGDRVTITCRASQDVSTAVAWYQQKPGKAPKLLIYSASFLYSGVPS
RFSGSGSGTDFTLTISSLQPEDFATYYCQQYLYHPATFGQGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC,
Avelumab Heavy Chain Sequence
SEQ ID NO: 8
EVQLLESGGGLVQPGGSLRLSCAASGFTFSSYIMMWVRQAPGKGLEWVSSIYPSGGITFY
ADTVKGRFTISRDNSKNTLYLQMNSLRAEDTAVYYCARIKLGTVTTVDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTOTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK,
Avelumab Light Chain Sequence
SEQ ID NO: 9
QSALTQPASVSGSPGQSITISCTGTSSDVGGYNYVSWYQQHPGKAPKLMIYDVSNRPSGV
SNRFSGSKSGNTASLTISGLQAEDEADYYCSSYTSSSTRVFGTGTKVTVLGQPKANPTVT
LFPPSSEELQANKATLVCLISDFYPGAVTVAWKADGSPVKAGVETTKPSKQSNNKYAASS
YLSLTPEQWKSHRSYSCQVTHEGSTVEKTVAPTECS,
MDX1105 Heavy Chain Sequence
SEQ ID NO: 10
QVQLVQSGAEVKKPGSSVKVSCKTSGDTFSTYAISWVRQAPGQGLEWMGGIIPIFGKAHYAQ
KFQGRVTITADESTSTAY
MELSSLRSEDTAVYFCARKFHFVSGSPFGMDVWGQGTTVTVSSASTKGPSVFPLAPSSKSTS
GGTAALGCLVKDYFPEPV
TVSWNSGALTSGVHTFPAVLQSSGLYSLSSVVTVPSSSLGTQTYICNVNHKPSNTKVDKKVE
PKSCDKT,
MDX1105 Light Chain Sequence
SEQ ID NO: 11
EIVLTQSPATLSLSPGERATLSCRASQSVSSYLAWYQQKPGQAPRLLIYDASNRATGIPARF
SGSGSGTDFTLTISSLEP
EDFAVYYCQQRSNWPTFGQGTKVEIKRTVAAPSVFIFPPSDEQLKSGTASVVCLLNNFYPRE
AKVQWKVDNALQSGNSQE
SVTEQDSKDSTYSLSSTLTLSKADYEKHKVYACEVTHQGLSSPVTKSFNRGEC,
Pembrolizumab - DB09037 >Heavy Chain Sequence
SEQ ID NO: 12
QVQLVQSGVEVKKPGASVKVSCKASGYTFTNYYMYWVRQAPGQGLEWMGGINPSNGGTNF
NEKFKNRVTLTTDSSTTTAYMELKSLQFDDTAVYYCARRDYRFDMGFDYWGQGTTVTVSS
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK,
Pembrolizumab Light Chain Sequence
SEQ ID NO: 13
EIVLTQSPATLSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIKRTVAAPSVE
IFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC,
Nivolumab Heavy Chain Sequence
SEQ ID NO: 14
QVQLVESGGGVVQPGRSLRLDCKASGITFSNSGMHWVRQAPGKGLEWVAVIWYDGSKRYY
ADSVKGRFTISRDNSKNTLFLQMNSLRAEDTAVYYCATNDDYWGQGTLVTVSSASTKGPS
VFPLAPCSRSTSESTAALGCLVKDYFPEPVTVSWNSGALTSGVHTFPAVLQSSGLYSLSS
VVTVPSSSLGTKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTC
LVKGFYPSDIAVEWESNGQPENNYKTTPPVLDSDGSFFLYSRLTVDKSRWQEGNVFSCSV
MHEALHNHYTQKSLSLSLGK,
Nivolumab Light Chain Sequence
SEQ ID NO: 15
EIVLTQSPATLSLSPGERATLSCRASQSVSSYLAWYQQKPGQAPRLLIYDASNRATGIPA
RFSGSGSGTDFTLTISSLEPEDFAVYYCQQSSNWPRTFGQGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC,
Cemiplimab Heavy Chain Sequence
SEQ ID NO: 16
EVQLLESGGVLVQPGGSLRLSCAASGFTFSNFGMTWVRQAPGKGLEWVSGISGGGRDTYF
ADSVKGRFTISRDNSKNTLYLQMNSLKGEDTAVYYCVKWGNIYFDYWGQGTLVTVSSAST
KGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFLGGPSVFLF
PPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTVDKSRWQEGNVF
SCSVMHEALHNHYTQKSLSLSLGK,
Cemiplimab Light Chain Sequence
SEQ ID NO: 17
DIQMTQSPSSLSASVGDSITITCRASLSINTFLNWYQQKPGKAPNLLIYAASSLHGGVPS
RFSGSGSGTDFTLTIRTLQPEDFATYYCQQSSNTPFTFGPGTVVDFRRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC,
MAGE A4 peptide
SEQ ID NO: 18
GVYDGREHTV,
(CD8α)CDRs bold underlined, signal sequence italic underlined
SEQ ID NO: 19
MALPVTALLLPLALLLHAARPSQFRVSPLDRTWNLGETVELKCQ [symbol] CSWLFQPRG
AAASPTFLL [symbol] AAEGLDTQRFSGKRLGDTFVLTLSDFRRENEGYYFCSA [symbol] Y
FSHFVPVFLPAKPTTTPAPRPPTPAPTIASQPLSLRPEACRPAAGGAVHTRGLDFACDIYIW
APLAGTCGVLLLSLVITLYCNHRNRRRVCKCPRPVVKSGDKPSLSARYV;
(CD8α)
SEQ ID NO: 20
ATGGCCTTACCAGTGACCGCCTTGCTCCTGCCGCTGGCCTTGCTGCTCCACGCCGCCAGGCC
GAGCCAGTTCCGGGTGTCGCCGCTGGATCGGACCTGGAACCTGGGCGAGACAGTGGAGCTGA
AGTGCCAGGTGCTGCTGTCCAACCCGACGTCGGGCTGCTCGTGGCTCTTCCAGCCGCGCGGC
GCCGCCGCCAGTCCCACCTTCCTCCTATACCTCTCCCAAAACAAGCCCAAGGCGGCCGAGGG
GCTGGACACCCAGCGGTTCTCGGGCAAGAGGTTGGGGGACACCTTCGTCCTCACCCTGAGCG
ACTTCCGCCGAGAGAACGAGGGCTACTATTTCTGCTCGGCCCTGAGCAACTCCATCATGTAC
TTCAGCCACTTCGTGCCGGTCTTCCTGCCAGCGAAGCCCACCACGACGCCAGCGCCGCGACC
ACCAACACCGGCGCCCACCATCGCGTCGCAGCCCCTGTCCCTGCGCCCAGAGGCGTGCCGGC
CAGCGGCGGGGGGCGCAGTGCACACGAGGGGGCTGGACTTCGCCTGTGATATCTACATCTGG
GCGCCCTTGGCCGGGACTTGTGGGGTCCTTCTCCTGTCACTGGTTATCACCCTTTACTGCAA
CCACAGGAACCGAAGACGTGTTTGCAAATGTCCCCGGCCTGTGGTCAAATCGGGAGACAAGC
CCAGCCTTTCGGCGAGATACGTCGGTTCAAGAGCTAAAAGAAGTGGTAGTGGTGCCCCTGTG
A;
(MAGE A4 TCR α chain) CDRs bold underlined
SEQ ID NO: 21
MKKHLTTFLVILWLYFYRGNGKNQVEQSPQSLIILEGKNCTLQCNYT [symbol] LRWYKQDTG
RGPVSLTI [symbol] TKSNGRYTATLDADTKQSSLHITASQLSDSASYI [symbol] [symbol] [symbol]
GAGTQVVVTPDIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNFQ
NLSVIGFRILLLKVAGFNLLMTLRLWSSGSRAKR;
(MAGE A4 TCR α chain coding sequence)
SEQ ID NO: 22
ATGAAGAAGCACCTGACCACCTTTCTCGTGATCCTGTGGCTGTACTTCTACCGGGGCAACGG
CAAGAACCAGGTGGAACAGAGCCCCCAGAGCCTGATCATCCTGGAAGGCAAGAACTGCACCC
TGCAGTGCAACTACACCGTGTCCCCCTTCAGCAACCTGCGGTGGTACAAGCAGGACACCGGC
AGAGGCCCTGTGTCCCTGACCATCCTGACCTTCAGCGAGAACACCAAGAGCAACGGCCGGTA
CACCGCCACCCTGGACGCCGATACAAAGCAGAGCAGCCTGCACATCACCGCCAGCCAGCTGA
GCGATAGCGCCAGCTACATCTGCGTGGTGTCCGGCGGCACAGACAGCTGGGGCAAGCTGCAG
TTTGGCGCCGGAACACAGGTGGTCGTGACCCCCGACATCCAGAACCCTGACCCTGCCGTGTA
CCAGCTGCGGGACAGCAAGAGCAGCGACAAGAGCGTGTGCCTGTTCACCGACTTCGACAGCC
AGACCAACGTGTCCCAGAGCAAGGACAGCGACGTGTACATCACCGACAAGACCGTGCTGGAC
ATGCGGAGCATGGACTTCAAGAGCAATAGCGCCGTGGCCTGGTCCAACAAGAGCGACTTCGC
CTGCGCCAACGCCTTCAACAACAGCATTATCCCCGAGGACACATTCTTCCCAAGCCCCGAGA
GCAGCTGCGACGTCAAGCTGGTGGAAAAGAGCTTCGAGACAGACACCAACCTGAACTTCCAG
AACCTGAGCGTGATCGGCTTCAGAATCCTGCTGCTGAAGGTGGCCGGCTTCAACCTGCTGAT
GACCCTGAGACTGTGGTCCAGCGGCAGCCGGGCCAAGAGA;
(MAGE A4 TCR β chain) CDRs bold underlined
SEQ ID NO: 23
MASLLFFCGAFYLLGTGSMDADVTQTPRNRITKTGKRIMLECSQT [symbol] MYWYRQDPGLGL
RLIYY [symbol] INKGEISDGYSVSRQAQAKFSLSLESAIPNQTALYF [symbol] [symbol] G
PGTRLTVLEDLKNVFPPEVAVFEPSEAEISHTQKATLVCLATGFYPDHVELSWWVNGKEVHS
GVSTDPQPLKEQPALNDSRYCLSSRLRVSATFWQNPRNHFRCQVQFYGLSENDEWTQDRAKP
VTQIVSAEAWGRADCGFTSESYQQGVLSATILYEILLGKATLYAVLVSALVLMAMVKRKDSR
G;
(MAGE A4 TCR β chain coding sequence)
SEQ ID NO: 24
ATGGCCAGCCTGCTGTTCTTCTGCGGCGCCTTCTACCTGCTGGGCACCGGCTCTATGGATGC
CGACGTGACCCAGACCCCCCGGAACAGAATCACCAAGACCGGCAAGCGGATCATGCTGGAAT
GCTCCCAGACCAAGGGCCACGACCGGATGTACTGGTACAGACAGGACCCTGGCCTGGGCCTG
CGGCTGATCTACTACAGCTTCGACGTGAAGGACATCAACAAGGGCGAGATCAGCGACGGCTA
CAGCGTGTCCAGACAGGCTCAGGCCAAGTTCAGCCTGTCCCTGGAAAGCGCCATCCCCAACC
AGACCGCCCTGTACTTTTGTGCCACAAGCGGCCAGGGCGCCTACGAGGAGCAGTTCTTTGGC
CCTGGCACCCGGCTGACAGTGCTGGAAGATCTGAAGAACGTGTTCCCCCCAGAGGTGGCCGT
GTTCGAGCCTTCTGAGGCCGAAATCAGCCACACCCAGAAAGCCACACTCGTGTGTCTGGCCA
CCGGCTTCTACCCCGACCACGTGGAACTGTCTTGGTGGGTCAACGGCAAAGAGGTGCACAGC
GGCGTGTCCACCGATCCCCAGCCTCTGAAAGAACAGCCCGCCCTGAACGACAGCCGGTACTG
CCTGAGCAGCAGACTGAGAGTGTCCGCCACCTTCTGGCAGAACCCCAGAAACCACTTCAGAT
GCCAGGTGCAGTTTTACGGCCTGAGCGAGAACGACGAGTGGACCCAGGACAGAGCCAAGCCC
GTGACACAGATCGTGTCTGCCGAAGCTTGGGGGCGCGCCGATTGTGGCTTTACCAGCGAGAG
CTACCAGCAGGGCGTGCTGAGCGCCACCATCCTGTACGAGATCCTGCTGGGAAAGGCCACAC
TGTACGCCGTGCTGGTGTCTGCCCTGGTGCTGATGGCCATGGTCAAGCGGAAGGACAGCCGG
GGC;
(MAGE A4 TCR α chain variable region)136AA - CDRs bold
underlined
SEQ ID NO: 25
MKKHLTTFLVILWLYFYRGNGKNQVEQSPQSLIILEGKNCTLQCNYT [symbol] LRWYKQDTG
RGPVSLTI [symbol] TKSNGRYTATLDADTKQSSLHITASQLSDSASYI [symbol] [symbol] [symbol]
GAGTQVVVTPD;
(MAGE A4 TCR β chain variable region)133AA - CDRs bold
underlined
SEQ ID NO: 26
MASLLFFCGAFYLLGTGSMDADVTQTPRNRITKTGKRIMLECSQT [symbol] MYWYRQDPGLGL
RLIYY [symbol] INKGEISDGYSVSRQAQAKFSLSLESAIPNQTALYF [symbol] [symbol] G
PGTRLTVLE;
CDR1 MAGE A4 TCR α chain, (residues 48-53)
SEQ ID NO: 27
VSPFSN;
CDR2 MAGE A4 TCR α chain, (residues 71-76)
SEQ ID NO: 28
LTFSEN;
CDR3 MAGE A4 TCR α chain, (residues 111-125)
SEQ ID NO: 29
CVVSGGTDSWGKLQF;
CDR1 MAGE A4 TCR β chain, (residues 46 - 50)
SEQ ID NO: 30
KGHDR;
CDR2 MAGE A4 TCR β chain, (residues 68-73)
SEQ ID NO: 31
SFDVKD;
CDR3 MAGE A4 TCR β chain, (residues 110 - 123)
SEQ ID NO: 32
CATSGQGAYEEQFF;
CDR1 CD8α (residues 45-53)
SEQ ID NO: 33
VLLSNPTSG;
CDR2 CD8α (residues 72-79)
SEQ ID NO: 34
YLSQNKPK;
CDR3 CD8α (residues 118-123)
SEQ ID NO: 35
LSNSIM;
MAGE A4
SEQ ID NO: 36
MSSEQKSQHC KPEEGVEAQE EALGLVGAQA PTTEEQEAAV SSSSPLVPGT
LEEVPAAESA GPPQSPQGAS ALPTTISFTC WRQPNEGSSS QEEEGPSTSP
DAESLFREAL SNKVDELAHF LLRKYRAKEL VTKAEMLERV IKNYKRCFPV
IFGKASESLK MIFGIDVKEV DPASNTYTLV TCLGLSYDGL LGNNQIFPKT
GLLIIVLGTI AMEGDSASEE EIWEELGVMG VYDGREHTVY GEPRKLLTQD
WVQENYLEYR QVPGSNPARY EFLWGPRALA ETSYVKVLEH VVRVNARVRI
AYPSLREAAL LEEEEGV,

### EXAMPLES

### Example 1

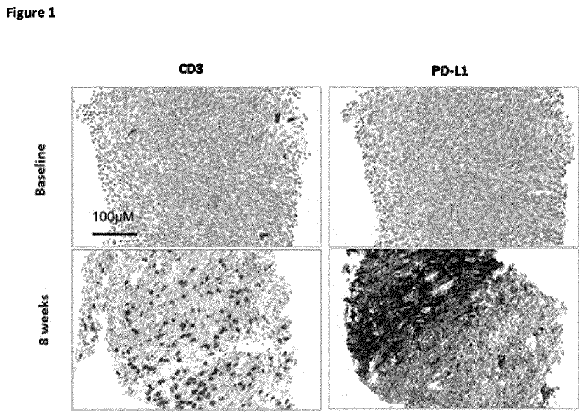

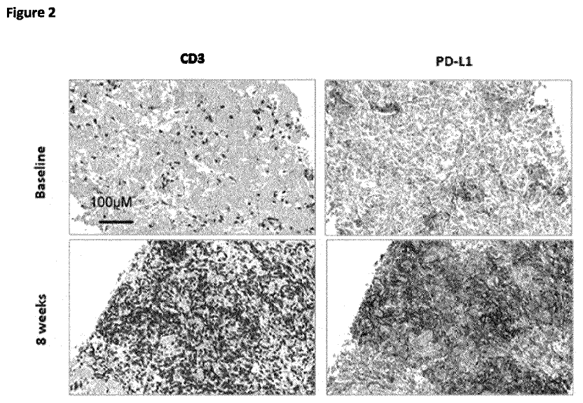

Using tissue biopsy samples taken before and after T-cell infusion (SPEAR T-cells i.e. comprising subject T-cells engineered with a heterologous TCR specific to MAGE A4 cancer testis antigen) from a subject synovial sarcoma patient (tumour expressing MAGE A4) we have shown that prior to the infusion there was no PD-L1 expression in the tissue. The patient was responsive to SPEAR T-cell treatment. Post SPEAR T-cell infusion, there was an increase in measured CD3 (part of the T cell receptor (TCR) complex on a mature T lymphocyte) expressed in the tissue signaling T-cell infiltration and the presence of the gene-modified T-cells. There was additionally an observed induction of PD-L1 in the tumor associated with the T-cell infiltration, FIG. 1. Data from biopsies from a subject ovarian cancer patient who was non-responsive to SPEAR T-cell treatment demonstrated that PD-L1 expression was positive in the pre-infusion biopsy and that following SPEAR T-cell infusion there was an increase in T-cell/gene-modified T-cell infiltration (indicated by CD3) and an observed associated stronger induction of PD-L1 in the tumor, FIG. 2.

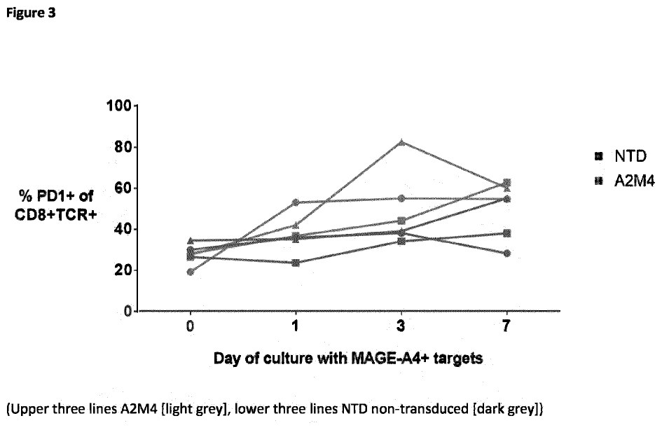

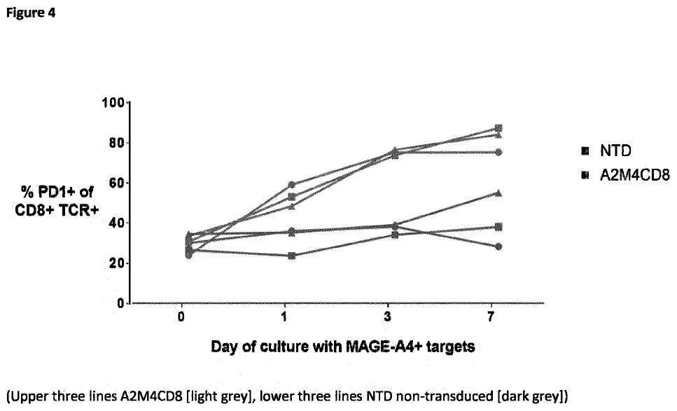

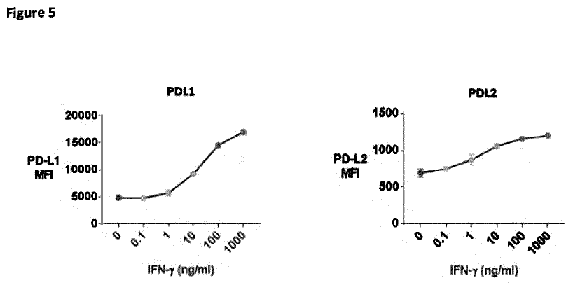

We have shown that exposure of gene-modified T-cells expressing heterologous TCR to MAGE-A4 upregulate PD-1 expression upon stimulation with the target antigen MAGE-A4 in comparison to non-transduced (NTD) T-cells not having the heterologous TCR, FIG. 3. The same effect is seen in gene-modified T-cells expressing heterologous MAGE-A4 and heterologous CD8, FIG. 4. The data indicates that the activity of the engineered T-cells could be enhanced by blocking the PD1/PD-L1 interaction. We have also shown that cancer cell lines upregulate PD-L1 in response to interferon, FIG. 5. The data of FIG. 5 demonstrates that what is seen clinically in patients is also seen in the in-vitro cancer cell assay system. As part of the immune response to cancer T-cells produce interferon which has an anti-tumour effect through the JAK/STAT pathway of the target cell. In patients the PD-L1 upregulation response of tumour to interferon represents part of the immune evasion adaptations of tumour cells.

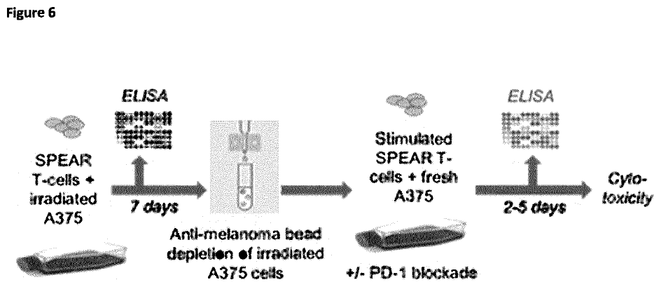

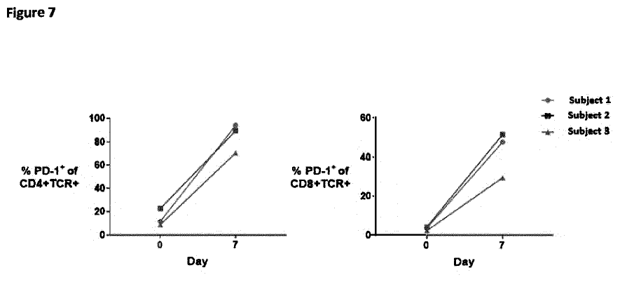

We have further examined the effect of PD-1 blockade on the cytotoxicity and effector cytokine production by gene-modified T-cells expressing heterologous TCR to MAGE-A4 (SPEAR T cells) using a pre-stimulation protocol and ELISA assay for cytokines. T-cell pre-activation was performed as set out in FIG. 6, in which gene-modified T-cells expressing heterologous TCR to MAGE-A4 (also performed for gene-modified T-cells expressing both heterologous MAGE-A4 and heterologous CD8) are stimulated with target expressing tumour cells (irradiated A375 MAGE-A4⁺ melanoma cells), cultured and separated from the cancer cells prior to re-stimulation in the presence of absence of PD-1 blockade with anti-PD-1 antibody. The data of FIG. 7 shows that the expression of PD-1 on the pre-activated T-cells over the period of the initial stimulation of 7 days increases for both CD4+ and CD8+ cells over each population.

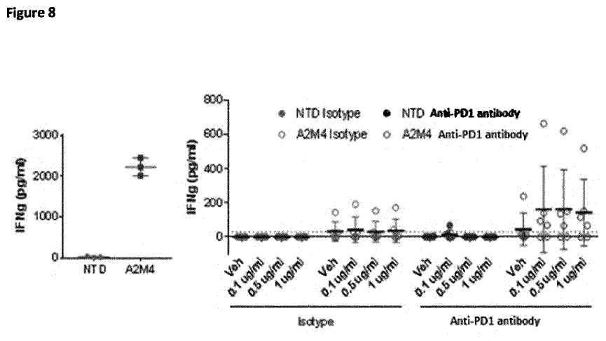

Using the pre-stimulation model we have shown that IFN-gamma, IL-2 and granzyme B are produced during the initial stimulation in both engineered MAGE-A4 TCR and engineered MAGE-A4 TCR+CD8 T-cells during the initial stimulation but this ability is lost after this pre-activation step on re-stimulation. However, re-stimulation in the presence of anti-PD-1 antibody partially recovers this cytokine activity function and restores IFN gamma and granzyme B but not IL-2 production for both engineered MAGE-A4 TCR and engineered MAGE-A4 TCR+CD8 T-cells, FIG. 8 shows data for MAGE-A4 TCR and IFN-gamma. In conclusion we have shown the ability to rescue exhausted engineered T-cell cytokine function by combination with anti-PD-1 antibody.

### Example 2, In Vivo Tumour Model

We have further tested the combination in a NSG strain mouse A375 (melanoma) xenograft model using a dosing of 1×10⁶ transduced (ADP-A2M4) MAGE-A4 TCR T cells; 10 mg/kg intra peritoneal q4d (twice a day for four days) anti-PD-1 antibody pembrolizumab (Keytruda) and the effect on tumour volume recorded according to the following protocol. 72 tumour bearing mice are randomised into 9 groups of n=8, and treated as per Table 1 below.

TABLE 1

| | Treatment 1 | Treatment 2 | Scheduled |
|---|---|---|---|
| Group | Dosing | Dosing | euthanasia |
| 01 | Isotype control (IgG4) | — | Day 21 |
| (n = 8) | Q4D from Day 0, i.p. | | |
| 02 | Pembrolizumab | — | Day 21 |
| (n = 8) | Q4D from Day 0, i.p. | | |
| | 10 mg/kg | | |
| 03 | NTD T-cells | — | Day 21 |
| (n = 8) | Day 0 only, i.v. | | |
| | (1 × 10\hat{ }6cells) | | |
| 04 | NTD T-cells | Pembrolizumab | Day 21 |
| (n = 8) | Day 0 only, i.v. | Q4D from Day 1, i.p. | |
| | (1 × 10\hat{ }6cells) | 10 mg/kg | |
| 05 | T-cells | Isotype control (IgG4) | Day 60 |
| (n = 8) | Day 0 only, i.v. | Q4D from Day 1, i.p. | |
| | (1 × 10\hat{ }6cells) | | |
| 06 | T-cells | Pembrolizumab | Day 60 |
| (n = 8) | Day 0 only, i.v. | Q4D from Day −1, i.p. | |
| | (1 × 10\hat{ }6cells) | 10 mg/kg | |
| 07 | T-cells | Pembrolizumab | Day 60 |
| (n = 8) | Day 0 only, i.v. | Q4D from Day 7, i.p. | |
| | (1 × 10\hat{ }6cells) | 10 mg/kg | |
| 08 | T-cells | Pembrolizumab | Day 60 |
| (n = 8) | Day 0 only, i.v. | Q4D from Day 14, i.p. | |
| | (1 × 10\hat{ }6cells) | 10 mg/kg | |
| 09 | T-cells | Pembrolizumab | Day 60 |
| (n=8) | Day 0 only, i.v. | Q4D from Day 21, i.p. | |
| | (1 × 10\hat{ }6cells) | 10 mg/kg | |

All animals in groups 1 and 2 will be administered either isotype control (group 1) or pembrolizumab (group 2) Q4D from Day 0 (i.e. Day 0, 4, 8, 12, 16, 20) via i.p. (maximum dosing volume via i.p. 10 ml/kg). No other treatment will be administered. (Day 0 is the day on which T-cells are administered as per groups 3 to 9).

All animals in groups 3 and 4 will receive a single administration of NTD T-cells on Day 0 via i.v. at a recommended volume of 5 ml/kg. No other treatment will be administered to animals in group 3. In addition, animals in group 4 will receive pembrolizumab Q4D from Day 1 (i.e. Day 1, 5, 9, 13, 17) via i.p. (maximum dosing volume via i.p. 10 ml/kg)

All animals in groups 5, 6, 7, 8 and 9 will receive a single administration of T-cells on Day 0 via i.v. at a recommended volume of 5 ml/kg and groups 5, 6, 7, 8 and 9 a second treatment as described below:
Group 5: isotype control Q4D from Day 1 (i.e. Day 1, 5, 9, 13, 17, 21, 25, 29, 33, 37, 41, 45, 49, 53, 57) via i.p. (maximum dosing volume via i.p. 10 ml/kg)
Group 6: pembrolizumab Q4D from Day −1 (i.e. Day −1, 3, 7, 11, 15, 19, 23, 27, 31, 35, 39, 43, 47, 51, 55, 59) via i.p. (maximum dosing volume via i.p. 10 ml/kg)
Group 7: pembrolizumab Q4D from Day 7 (i.e. Day 7, 11, 15, 19, 23, 27, 31, 35, 39, 43, 47, 51, 55, 59) via i.p. (maximum dosing volume via i.p. 10 ml/kg)
Group 8: pembrolizumab Q4D from Day 14 (i.e. Day 14, 18, 22, 26, 30, 34, 38, 42, 46, 50, 54, 58) via i.p. (maximum dosing volume via i.p. 10 ml/kg)
Group 9: pembrolizumab Q4D from Day 21 (i.e. Day 21, 25, 29, 33, 37, 41, 45, 49, 53, 57) via i.p. (maximum dosing volume via i.p. 10 ml/kg)

In all cases tumour volumes are measured with callipers 3 times per week and animals weighed 3 times per week from initiation of treatment.

### Example 3, Clinical Investigation

An investigation was designed to investigate combination treatment with Pembrolizumab with ADP-A2M4 (SPEAR T-cell, T-cell engineered to express MAGE A4 specific TCR) for the treatment of patients with recurrent or metastatic HNSCC in which the subject has received no prior systemic therapy for metastatic disease or subject has disease progression on or after platinum containing chemotherapy. Disease may be histologically or cytogenetically confirmed and/or measurable disease according to RECIST v1.1.

The study is a single arm Phase II study (n=10 patients) in advanced/recurrent Head and neck squamous cell carcinoma (HNSCC) with checkpoint inhibitor naïve patients in which the subject has received 0-1 prior lines of platinum containing systemic therapy for metastatic disease or in which the subject is checkpoint inhibitor naïve at screening prior to treatment and is due to start pembrolizumab treatment, or has recently started pembrolizumab treatment for advanced disease (having received 1-2 doses). Subjects are determined as PD-L1+, HLA-A2+, MAGE-A4 expression: 2+/3+ in ≥30% cells by IHC [i.e. tumour shows MAGE-A4 expression defined as ≥30% of tumour cells that are ≥2+ by IHC (ImmunoHistoChemistry)]. The primary endpoint for measure is ORR by RECIST v1.1. Historic response rates of standard of care PD-1 therapy, pembrolizumab monotherapy, in advanced/recurrent Head and neck squamous cell carcinoma (HNSCC) produces an ORR=19%.

Prior to initiating pembrolizumab treatment the subject is apheresed to obtain the T-cell population, the cells are subsequently transduced with the ADP-A2M4 “SPEAR” TCR specific for MAGE A4 antigen (particularly the specific MAGE A4 antigenic peptide SEQ ID NO:18) and the cells expanded and cryopreserved for later use. The subject is thereafter treated with up to 3 doses of pembrolizumab (200 mg IV infusion over 30 mins every 3 weeks) and then scanned for disease status, for example measurable disease according to RECIST v1.1. Subjects with progressive disease or stable disease go forward for a lymphodepletion regimen, fludarabine (30 mg/m²/day for 4 days) and cyclophosphamide (600 mg/m²/day for 3 days) and SPEAR T cell infusion: ADP-A2M4 dosing of 1 billion to 10 billion transduced cells with no dose escalation and continue pembrolizumab (200 mg IV infusion over 30 mins every 3 weeks) until disease progression or until 24 months whichever is the earlier. Subjects scanned and determined to have complete or partial response do not undergo lymphodepletion and cell infusion but continue pembrolizumab (200 mg IV infusion over 30 mins every 3 weeks) until disease progression or until 24 months whichever is the earlier, after which they may then undergo lymphodepletion and cell infusion and combination pembrolizumab administration. Optionally apheresis is to occur prior to start of pembrolizumab. First dose of pembrolizumab following cell infusion may be on day 17 or 22 following the cell infusion.

For the present study the subject exclusion criteria include: (a) HLA-A*02:05 positive in either allele. (b) HLA-A*02:07 (and alleles having the same protein sequence in the antigen binding domains as A*02:07) or any A*02 null allele (designated with an “N” suffix, e.g. A*02:32N) as the sole HLA-A*02 allele (e.g. a subject with HLA alleles A*02:04 and A*02:07 is eligible), (c) CNS metastases, (d) any prior checkpoint inhibitor therapy, (e) any prior cell therapy, (f) if received chemotherapy, wash out period required before leukapheresis or LD is 3 weeks.

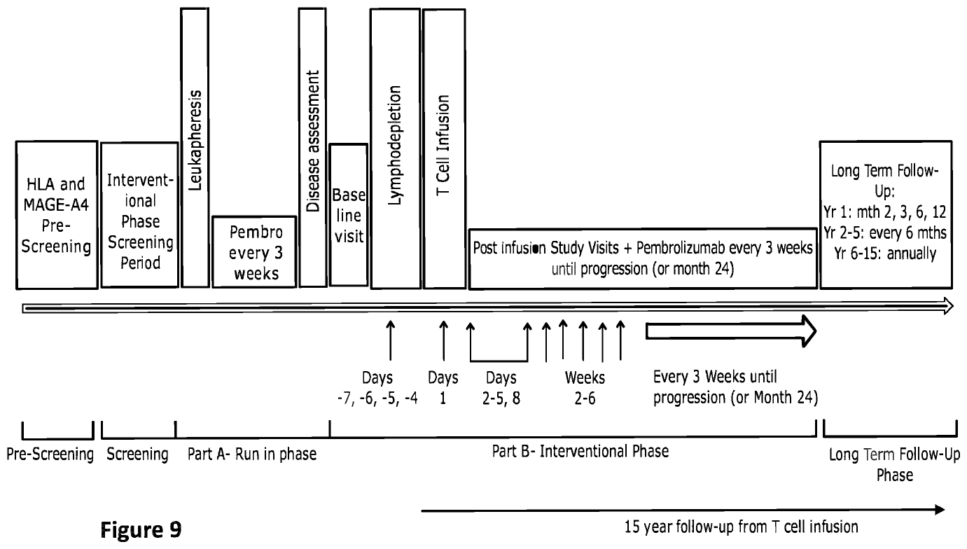

The primary endpoint for measure is ORR by RECIST v1.1. The treatment scheme is presented in FIG. 9.

What is claimed is:

1. A method of treating, preventing or delaying the progression of cancer and/or tumour in a subject comprising administering to the subject a treatment regimen comprising an effective amount of a PD-1 axis binding antagonist and a population of modified immunoresponsive cells expressing or presenting a heterologous TCR.

2. The method of claim 1, wherein the PD-1 axis binding antagonist is selected from the group consisting of
(a) a PD-1 binding antagonist,
(b) a PD-L1 binding antagonist,
(c) a PD-L2 binding antagonist.

3. The method of claim 2, wherein:
(i) the PD-1 axis binding antagonist binds to PD-1 and/or binds to SEQ ID NO: 1, optionally wherein the PD-1 binding antagonist inhibits the binding of PD-1 to its ligand binding partners, preferably wherein the PD-1 binding antagonist inhibits the binding of PD-1 to PD-L1 and/or PDL2;
(ii) the PD-1 axis binding antagonist binds to PD-L1 and/or binds to SEQ ID NO: 2, optionally wherein the PD-L1 binding antagonist inhibits the binding of PD-L1 to its ligand binding partners, preferably wherein the PD-L1 binding antagonist inhibits the binding of PD-L1 to one or more of PD-1, CD80 and B7-1.

4. The method of claim 3, wherein the PD-1 or PD-L1 binding antagonist is an antibody; optionally wherein:
(a) the anti-PD-L1 antibody inhibits binding between PD-L1 and PD-1 and/or between PD-L1 and B7-1,
(b) the anti-PD-L1 antibody inhibits PD-L1 on the cancer cell surface from transducing a signal to the intracellular pathway,
(c) the anti-PD-1 antibody inhibits binding between PD-L1 and PD-1 and/or between PD-L2 and PD-1,
(d) the anti-PD-1 antibody inhibits PD-1 on the T cell surface from transducing a signal to the intracellular pathway.

5. The method of claim 4, wherein:
(1) the PD-L1 binding antagonist is selected from (a) Durvalumab, Imfinzi or MEDI4736, (b) Atezolizumab, Tecentriq or MPDL3280A, (c) Avelumab, Bavencio or MSB0010718C, or (d) MDX-1105, BMS-936559,
optionally wherein the PD-L1 binding antagonist is an antibody comprising:
(i) a heavy chain comprising SEQ ID NO:4, and a light chain comprising SEQ ID NO:5, or variable regions thereof or CDRs thereof,
(ii) a heavy chain comprising SEQ ID NO:6, and a light chain comprising SEQ ID NO:7, or variable regions thereof or CDRs thereof,
(iii) a heavy chain comprising SEQ ID NO:8, and a light chain comprising SEQ ID NO:9, or variable regions thereof or CDRs thereof, or
(iv) a heavy chain comprising SEQ ID NO:10, and a light chain comprising SEQ ID NO:11, or variable regions thereof or CDRs thereof,
and further optionally wherein the antibody is monoclonal, human or humanised and is a full length or antigen-binding fragment thereof, Fv, Fab, Fab′, Fab′-SH, F(ab′)2; diabody; linear antibody; single-chain antibody molecule, scFv; or
(2) the PD-1 binding antagonist is selected from (a) Pembrolizumab, Keytruda, Lambrolizumab or MK-3475, (b) Cemiplimab, Libtayo, or REGN-2810, or (c) BMS/ONO, Nivolumab, Opdivo, ONO-4538, BMS-936558 or MDX1106,
optionally wherein the PD-1 binding antagonist is an antibody comprising:
(i) a heavy chain comprising SEQ ID NO:12, and a light chain comprising SEQ ID NO:13, or variable regions thereof or CDRs thereof,
(ii) a heavy chain comprising SEQ ID NO:14, and a light chain comprising SEQ ID NO:15, or variable regions thereof or CDRs thereof, or
(iii) a heavy chain comprising SEQ ID NO:16, and a light chain comprising SEQ ID NO: 17, or variable regions thereof or CDRs thereof,
and further optionally wherein the antibody is monoclonal, human or humanised and is a full length or antigen-binding fragment thereof, Fv, Fab, Fab′, Fab′-SH, F(ab′)2; diabody; linear antibody; single-chain antibody molecule, scFv.

6. The method of claim 2, wherein the PD-1 axis binding antagonist is a PD-L2 binding antagonist and/or binds to SEQ ID NO: 3, optionally wherein the PD-L2 binding antagonist is an antibody or an immunoadhesin.

7. The method of claim 1, wherein (a) the heterologous TCR binds or specifically binds to a cancer and/or tumour antigen or peptide antigen thereof, or (b) the heterologous TCR binds or specifically binds to a cancer and/or tumour antigen or peptide antigen thereof associated with a cancerous condition and/or presented by tumour or cancer cell or tissue; optionally wherein:
(i) the cancer and/or tumour antigen or peptide antigen thereof is complexed with a peptide presenting molecule, optionally major histocompatibility complex (MHC) or human leukocyte antigen (HLA), optionally class I or class II, optionally wherein the peptide is complexed with HLA-A2 or HLA-A*02, or HLA-A*0201;
(ii) the cancer and/or tumour antigen or peptide antigen thereof is a cancer-testis antigen;
(iii) the cancer and/or tumour antigen or peptide antigen thereof is selected from any of; NY-ESO-1, MART-1 (melanoma antigen recognized by T cells), WT1 (Wilms tumor 1), gp100 (glycoprotein 100), tyrosinase, PRAME (preferentially expressed antigen in melanoma), p53, HPV-E6/HPV-E7 (human papillomavirus), HBV, TRAIL, DR4, Thyroglobin, TGFBII frameshift antigen, LAGE-1A, KRAS, CMV (cytomegalovirus), CEA (carcinoembryonic antigen), AFP (α-fetoprotein), MAGE-AL MAGE-A2, MAGE-A3, MAGE-A4, MAGE-A6, MAGE-A8, and MAGE-A9, MAGE-A10, or MAGE-A12, or peptide antigen thereof;
(iv) the cancer and/or tumour antigen or peptide antigen is MAGE-A4 or peptide thereof, preferably the sequence GVYDGREHTV or SEQ ID NO: 18; and/or
(v) the heterologous TCR binds specifically and/or selectively to the cancer and/or tumour antigen or peptide antigen thereof and/or the peptide presenting molecule and/or complex thereof.

8. The method of claim 1, wherein:
(a) the heterologous TCR comprises a TCR alpha chain variable domain and a TCR beta chain variable domain, wherein:
(i) the alpha chain variable domain comprises CDRs having the sequences
VSPFSN (αCDR1), SEQ ID NO:27 or amino acids 48-53 of SEQ ID NO:21,
LTFSEN (αCDR2), SEQ ID NO:28 or amino acids 71-76 of SEQ ID NO:21, and
CVVSGGTDSWGKLQF (αCDR3), SEQ ID NO:29 or amino acids 111-125 of SEQ ID NO:21, and
(ii) the beta chain variable domain comprises CDRs having the sequences
KGHDR (βCDR1), SEQ ID NO:30 or amino acids 46-50 of SEQ ID NO:23,
SFDVKD (βCDR2), SEQ ID NO:31 or amino acids 68-73 of SEQ ID NO:23, and
CATSGQGAYEEQFF (βCDR3), SEQ ID NO:32 or amino acids 110-123 of SEQ ID NO:23;
or sequences having at least 80% sequence identity thereto; and/or
b) the heterologous TCR comprises a TCR in which the alpha chain variable domain comprises an amino acid sequence that has at least 80%, identity to SEQ ID NO:25 and/or the beta chain variable domain comprising an amino acid sequence that has at least 80% identity to SEQ ID NO:26.

9. The method according to claim 1, wherein the population of modified immunoresponsive cells expressing or presenting a heterologous TCR further expresses or presents a heterologous co-receptor, optionally wherein the co-receptor is a CD8 co-receptor, optionally wherein:
(i) the heterologous CD8 co-receptor is a heterodimer or a homodimer, a CD8ab heterodimer or a CD8αα homodimer; and/or
(ii) the heterologous CD8 co-receptor comprises;
(a) a CDR 1 of at least 80% sequence identity to amino acid sequence VLLSNPTSG, SEQ ID NO:33, CDR 2 of at least 80% sequence identity to amino acid sequence YLSQNKPK SEQ ID NO:34 and CDR 3 of at least 80% sequence identity amino acid sequence LSNSIM SEQ ID NO:35,
(b) a CDR 1 of amino acid sequence VLLSNPTSG, SEQ ID NO:33, CDR 2 of amino acid sequence YLSQNKPK SEQ ID NO:34 and CDR 3 of amino acid sequence LSNSIM SEQ ID NO:35,
(c) an amino acid sequence having at least 80% sequence identity to amino acids number 22 to 235 of SEQ ID NO: 19, or
(d) an amino acid sequence having 100% sequence identity to amino acids number 22 to 235 of sequence of SEQ ID NO: 19.

10. The method of claim 1, wherein:
(i) population of modified immunoresponsive cells expressing or presenting a heterologous TCR further expresses or presents a heterologous co-stimulatory ligand; optionally 4-1BBL, or CD80; and/or
(ii) the modified immunoresponsive cell is (a) a B cell, T cell or natural killer (NK) cell, (b) a T cell, optionally a CD4⁺ T cell or a CD8⁺ T cell.

11. The method of claim 1, wherein:
(i) the PD-1 axis binding antagonist and modified immunoresponsive cells are administered separately, sequentially or simultaneously;
(ii) the PD-1 axis binding antagonist is administered:
(a) prior to, simultaneous with or after the modified immunoresponsive cells,
(b) prior to and after the modified immunoresponsive cells, or
(c) simultaneously with and after the modified immunoresponsive cells.
(d) prior to and simultaneously with the modified immunoresponsive cells,
(e) after the modified immunoresponsive cells, or
(f) prior to and simultaneously with and after the modified immunoresponsive cells; and/or
(iii) the PD-1 axis binding antagonist and/or the modified immunoresponsive cells are administered continuously or intermittently.

12. The method of claim 1, wherein:
(i) the modified immunoresponsive cells are administered as a single dose;
(ii) the modified immunoresponsive cells are administered at a dose of between about 500 million to about 1 billion cells, about 2 billion to about 5 billion cells or about 6 billion to about 10 billion cells; or
(iii) the PD-1 axis binding antagonist is administered at a dose of:
(a) between about 1 to 9 or about 10 to 20 mg/kg,
(b) between about 3 to 5 mg/kg,
(c) between about 50 to 200 mg or about 300 to 500 mg, or
(d) about 200 mg,
optionally wherein the dose is a fixed dose.

13. The method of claim 1 wherein:
(i) the PD-1 axis binding antagonist is administered as
(a) a single dose in each of one or more dosing cycles,
(b) one or more doses in each of one or more dosing cycles,
(c) a single dose on the first day of each of one or more dosing cycles,
(d) one or more doses in each of one or more dosing cycles, at least one dose being on the first day of each cycle;
(ii) the dosing cycle is
(a) 14 to 17 days, 18 to 21 days, 22 to 24 days, 24 to 27 days, 28 to 30 days or 31 days,
(b) one week, two weeks, three weeks, four weeks or one month; and/or
(iii) the first dose of PD-1 axis binding antagonist after the modified immunoresponsive cells are administered is on either day 17, day 21 or day 22 after the immunoresponsive cells are administered.

14. The method of claim 1, wherein the PD-1 axis binding antagonist is administered as one or more doses on each of one or more dosing cycles, prior to administration of the modified immunoresponsive cells and is administered as one or more doses on each of one or more dosing cycles after administration of the modified immunoresponsive cells, optionally wherein:
(i) (a) the PD-1 axis binding antagonist is administered as one or more doses on each of one or more dosing cycles prior to administration of the modified immunoresponsive cells,
(b) the status of disease is determined in comparison to the status prior to PD-1 axis binding antagonist administration, wherein if stable disease or progressive disease is determined then,
(c) modified immunoresponsive cells are administered and the PD-1 axis binding antagonist is administered as one or more doses on each of one or more dosing cycles after the administration of the modified immunoresponsive cells, optionally wherein the PD-1 axis binding antagonist is administered for a specified period, preferably 24 months; or
(ii) (a) the PD-1 axis binding antagonist is administered as one or more doses on each of one or more dosing cycles prior to the administration of the modified immunoresponsive cells,
(b) the status of disease is determined in comparison to the status prior to PD-1 axis binding antagonist administration, wherein if complete response or partial response is determined then,
(c) the PD-1 axis binding antagonist is administered as one or more doses on each of one or more dosing cycles without administration of the modified immunoresponsive cells, optionally wherein, in step (c) stable disease or progressive disease is determined then,
(d) modified immunoresponsive cells are administered and the PD-1 axis binding antagonist is administered as one or more doses on each of one or more dosing cycles after the administration of the modified immunoresponsive cells or simultaneously with and after the administration of the modified immunoresponsive cells, wherein in step (c) or (d) the PD-1 axis binding antagonist is administered for the shorter of a specified period or determination of disease progression, optionally wherein the specified period is 24 months.

15. The method of claim 1, wherein the PD-1 axis binding antagonist and/or modified immunoresponsive cells are administered intravenously or by intravenous infusion.

16. The method of claim 1, wherein:
(i) the cancer is relapsed cancer or refractory cancer or recurrent cancer or locally recurrent cancer or metastatic cancer, non-resectable cancer or locally confined, cancer with no surgical or radiotherapy option or inoperable cancer;
(ii) the subject has relapsed cancer or refractory cancer or recurrent cancer or has locally recurrent cancer or metastatic cancer or locally confined or inoperable cancer;
(iii) the cancer is selected from; lung cancer, non-small cell lung cancer (NSCLC), metastatic or advanced NSCLC, squamous NSCLC, adenocarcinoma NSCLC, adenosquamous NSCLC, large cell NSCLC, ovarian cancer, gastric cancer, urothelial cancer, esophageal cancer, esophagogastric junction cancer (EGJ), melanoma, bladder cancer, head and neck cancer, head and neck squamous cell carcinoma (HNSCC), cancer of the oral cavity, cancer of the oropharynx, cancer of the hypopharynx, cancer of the throat, cancer of the larynx, cancer of the the tonsil, cancer of the tongue, cancer of the soft palate, cancer of the pharynx, synovial sarcoma, myxoid round cell liposarcoma (MRCLS), optionally wherein the cancer or tumour express a MAGE antigen or peptide antigen thereof and/or express PD-L1 or PD-L2 [optionally with CPS≥1], optionally MAGE-A4 antigen or peptide antigen thereof and/or express PD-L1 or PD-L2 [optionally with CPS≥1]; and/or
(iv) the cancer is selected from any one of breast cancer, metastatic breast cancer, liver cancer, renal cell carcinoma, synovial sarcoma, urothelial cancer or tumour, pancreatic cancer, colorectal cancer, metastatic stomach cancer, metastatic gastric cancer, metastatic liver cancer, metastatic ovarian cancer, metastatic pancreatic cancer, metastatic colorectal cancer, metastatic lung cancer, colorectal carcinoma or adenocarcinoma, lung carcinoma or adenocarcinoma, pancreatic carcinoma or adenocarcinoma, mucinous adenoma, ductal carcinoma of the pancreas, hematological malignancy, optionally wherein the cancer or tumour express a MAGE antigen or peptide antigen thereof and/or express PD-L1 or PD-L2 [optionally with CPS≥1], optionally MAGE-A4 antigen or peptide antigen thereof and/or express PD-L1 or PD-L2 [optionally with CPS≥1].

17. The method of claim 1 wherein the subject has not received prior cancer treatment.

18. The method of claim 1, wherein the subject has received prior cancer treatment and/or has failed to respond to prior cancer treatment, optionally wherein:
(a) the prior treatment comprises; systemic and/or local therapy, optionally any one or more of surgery, radiation therapy cryotherapy, laser therapy, topical therapy and/or systemic therapy, for example any one or more of chemotherapy, hormonal therapy, targeted drugs, or immunotherapy;
(b) the prior treatment comprises a PD-1 axis binding antagonist, PD-L1 binding antagonist or PD-1 binding antagonist, optionally wherein the PD-1 axis binding antagonist is an antibody;
(c) the prior treatment comprises an Epidermal Growth Factor Receptor Antagonist, optionally Cetuximab;
(d) the prior treatment comprises chemotherapy comprising a platinum compound, optionally selected from Lipoplatin, Cisplatin, Carboplatin, Oxaliplatin, Nedaplatin, Triplatin tetranitrate, Phenanthriplatin, Satraplatin, Picoplatin:
(e) the prior treatment comprises chemotherapy comprising a chemotherapeutic agent selected from, methotrexate, capecitabine, taxane, anthracycline, paclitaxel, docetaxel, paclitaxel protein bound particles, doxorubicine, epirubicine, 5-fluorouracil, cyclophosphamide, afatinib, vincristine, etoposide or combinations thereof; or
(f) the prior treatment comprises chemotherapy comprising a chemotherapeutic agent selected from, FEC: 5-fluorouracil, epirubicine, cyclophosphamide; FAC: 5-fluorouracil, doxorubicine, cyclophosphamide; AC: doxorubicine, cyclophosphamide; EC: epirubicine, cyclophosphamide.

19. The method of claim 18, wherein:
(i) the subject has not received prior treatment in recurrence less than or equal to 12 months since the last treatment or less than or equal to 6 months since the last treatment; or
(ii) the subject has not received any prior adjuvant therapy (surgery followed by radiation and/or chemotherapy) in recurrence less than or equal to 12 months since the last treatment or in recurrence less than or equal to 6 months since the last treatment.

20. The method of claim 1, wherein the treatment effectively extends or improves:
(a) the progression free survival,
(b) the time to progression,
(c) the duration of response,
(d) the overall survival,
(e) the objective response or objective response rate,
(f) the overall response or overall response rate,
(g) partial response or partial response rate,
(h) complete response or complete response rate,
(i) stable disease rate or median stable disease
(j) median progression free survival,
(k) median time to progression,
(l) median duration of response,
(m) median overall survival,
(n) median objective response or median objective response rate,
(o) median overall response or median overall response rate,
(p) median partial response or median partial response rate,
(q) median complete response or median complete response,
(r) median stable disease rate or median stable disease,
in comparison to treatment with PD-1 axis binding antagonist alone or modified immunoresponsive cells alone.


SEQUENCE LISTING (36 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 3 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 4 - protein, 451 aa, synthetic construct
  REGION 1..451: note = recombinant sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 215 aa, synthetic construct
  REGION 1..215: note = recombinant sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 448 aa, synthetic construct
  REGION 1..448: note = recombinant sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, synthetic construct
  REGION 1..214: note = recombinant sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 450 aa, synthetic construct
  REGION 1..450: note = recombinant sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 216 aa, synthetic construct
  REGION 1..216: note = recombinant sequence
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 10 - protein, 229 aa, synthetic construct
  REGION 1..229: note = recombinant sequence
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 11 - protein, 213 aa, synthetic construct
  REGION 1..213: note = recombinant sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 447 aa, synthetic construct
  REGION 1..447: note = recombinant sequence
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 218 aa, synthetic construct
  REGION 1..218: note = recombinant sequence
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 440 aa, synthetic construct
  REGION 1..440: note = recombinant sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 214 aa, synthetic construct
  REGION 1..214: note = recombinant sequence
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 444 aa, synthetic construct
  REGION 1..444: note = recombinant sequence
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 214 aa, synthetic construct
  REGION 1..214: note = recombinant sequence
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 10 aa, synthetic construct
  REGION 1..10: note = recombinant sequence
GVYDGREHTV

SEQ ID NO: 19 - protein, 235 aa, synthetic construct
  REGION 1..235: note = recombinant sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 20 - DNA, 745 bp, synthetic construct
  misc_feature 1..745: note = recombinant sequene
  source 1..745: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcggttcaagagctaaaagaagtggtagtggtgcccctgtga

SEQ ID NO: 21 - protein, 282 aa, synthetic construct
  REGION 1..282: note = recombinant sequence
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 22 - DNA, 846 bp, synthetic construct
  misc_feature 1..846: note = recombinant sequence
  source 1..846: mol_type = other DNA
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagaga

SEQ ID NO: 23 - protein, 311 aa, synthetic construct
  REGION 1..311: note = recombinant sequence
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 24 - DNA, 933 bp, synthetic construct
  misc_feature 1..933: note = recombinant sequence
  source 1..933: mol_type = other DNA
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

SEQ ID NO: 25 - protein, 136 aa, synthetic construct
  REGION 1..136: note = recombinant sequence
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPD

SEQ ID NO: 26 - protein, 133 aa, synthetic construct
  REGION 1..133: note = recombinant sequence
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLE

SEQ ID NO: 27 - protein, 6 aa, synthetic construct
  REGION 1..6: note = recombinant sequnce
VSPFSN

SEQ ID NO: 28 - protein, 6 aa, synthetic construct
  REGION 1..6: note = recombinant sequence
LTFSEN

SEQ ID NO: 29 - protein, 15 aa, synthetic construct
  REGION 1..15: note = recombinant sequence
CVVSGGTDSWGKLQF

SEQ ID NO: 30 - protein, 5 aa, synthetic construct
  REGION 1..5: note = recombinant sequence
KGHDR

SEQ ID NO: 31 - protein, 6 aa, synthetic construct
  REGION 1..6: note = recombinant sequene
SFDVKD

SEQ ID NO: 32 - protein, 14 aa, synthetic construct
  REGION 1..14: note = recombinant sequence
CATSGQGAYEEQFF

SEQ ID NO: 33 - protein, 9 aa, synthetic construct
  REGION 1..9: note = recombinant sequence
VLLSNPTSG

SEQ ID NO: 34 - protein, 8 aa, synthetic construct
  REGION 1..8: note = recombinant sequence
YLSQNKPK

SEQ ID NO: 35 - protein, 6 aa, synthetic construct
  REGION 1..6: note = recombinant sequence
LSNSIM

SEQ ID NO: 36 - protein, 317 aa, synthetic construct
  REGION 1..317: note = recombinant sequence
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV